(19) United States
(12) Patent Application Publication
(10) Pub. No.: US 2013/0211249 A1
(43) Pub. Date: Aug. 15, 2013

(54) DRUG ELUTING HYDROGELS FOR CATHETER DELIVERY

(71) Applicants: Bradley Powers Barnett, Baltimore, MD (US); Rex C. Yung, Towson, MD (US); Philippe Gailloud, Towson, MD (US)

(72) Inventors: Bradley Powers Barnett, Baltimore, MD (US); Rex C. Yung, Towson, MD (US); Philippe Gailloud, Towson, MD (US)

(73) Assignee: The Johns Hopkins University, Baltimore, MD (US)

(21) Appl. No.: 13/811,639

(22) PCT Filed: Jul. 22, 2011
(86) PCT No.: PCT/US2011/045098

§ 371 Date: Apr. 25, 2013

Related U.S. Application Data
(60) Provisional application No. 61/366,567, filed on Jul. 22, 2010.

(51) Int. Cl. A61M 25/00; A61M 5/00

(52) U.S. Cl. CPC A61M 25/003; A61M 5/007
USPC 600/435; 604/82; 604/508; 604/506

(57) ABSTRACT

The invention features catheters, hydrogel compositions, and methods that useful for the treatment of various conditions and diseases. The invention also provides kits and instructions for use.

### RELATED APPLICATIONS

This application claims the benefit of U.S. Provisional Application No. 61/366,567 filed Jul. 22, 2010, the entire contents of which is hereby incorporated by reference in its entirety.

### BACKGROUND OF THE INVENTION

Rapid advances in endoscopic instruments has enabled smaller luminal diameters enabling steering toward the periphery of the lung. Parallel with the development of improved videobronchoscopes and instruments that can be used for diagnostic and therapeutic purposes has been rapid advances in CT technology. The widespread availability of ever faster MDCT (multislice detector CT) scanners in most clinical centers has allowed and encouraged clinicians to incorporate sophisticated imaging, including three-dimensional multiplanar reconstructions (MPRs), volumetric reconstructions, and virtual bronchoscopic projections as part of the preprocedural planning or as real time image guidance on the entire spectrum of tracheobronchial diagnostic and interventional procedures.

Increasingly, pulmonary tracheobronchial interventions are also image guided for the placement of fiducial markers to guide adjuvant or definitive anticancer therapies, including conformal, intensity-modulated and focused external-beam radiotherapy, endobronchial high-dose brachytherapy, and direct intratumoral deposit of cytotoxic therapy.

In benign, but often progressive small airways and parenchymal lung diseases, the growing burden of chronic obstructive pulmonary disease (COPD) in an aging population, coupled with clinical benefits seen with lung volume reduction surgery (LVRS) in selected patients, have spurred greater interest in minimally invasive tracheobronchial placement of a variety of novel valves, plugs and glue to achieve the same effect through bronchoscopic lung volume reduction. However, effective delivery of hydrogels can be problematic.

The standard bronchoscope has an outer diameter of 4.9 to 5.5 mm at the distal tip and a working channel of 2.0 mm diameter and will accommodate accessories of up to 1.8 mm in diameter. Therapeutic bronchoscopes have an outer diameter of 5.8 to 6.4 mm at the distal tip and a working channel of 2.6 to 3.2 mm. At the other end of the size spectrum, bronchoscopes for pediatric indications have been made even smaller, with outer diameters between 3.5 mm down to 2.7 mm, but most of these ultrathin devices have operating channels of only 1.2 mm, which limits their suctioning and flushing capabilities and their usable accessories.

Other devices include expandable fluid-filled balloons for airway dilation. Dedicated balloons are used for bronchoplasty and include controlled radial expansion pulmonary balloons (3 cm long and 6 to 20 mm in diameter) and biliary balloons adapted for the same purpose (1 to 2 cm long and 6 to 10 mm in diameter). Also useful are guidewires: flexible j-tip and/or soft tip guidewires that can fit through a 2.0 mm channel. Minimum length is 150 cm to allow exchange through a 60 cm length bronchoscope. However, due to the viscosity of the above described hydrogels, and the restrictions associated with the above delivery devices, it is difficult to consistently effectuate its delivery to targeted locations like, for example, the lungs.

In view of the widespread applicability of this technology, there is a need to further develop improved compositions and devices for more targeted delivery of biomaterials and agents.

### SUMMARY OF THE INVENTION

As described below, the present invention features novel catheters, compositions, and methods for treating clinical disorders.

In aspects, the invention provides a novel catheter. In embodiments, the catheter contains a first delivery channel interdisposed within a second delivery channel. In related embodiments, the first delivery channel fluidly communicates a first component of a biocompatible material. In related embodiments, the second delivery channel fluidly communicates a second component of the biocompatible material.

In embodiments, a distal end of the first delivery channel is interposed within the second delivery channel. In related embodiments, the length of the first delivery channel is shorter than a distal end of the second delivery channel.

In embodiments, the catheter contains a mixing tip. In related embodiments, the first component and the second component are mixed between the distal end of the first delivery channel and the distal end of the second delivery channel.

In embodiments, the length of the first delivery channel is variable.

In embodiments, the length of the first delivery channel is fixed.

In embodiments, a proximal end of the first delivery channel and the proximal end of the second delivery channel are connected respectively to a syringe via flexible connections. In related embodiments, the syringe contains a barrel having a first and second chamber. In related embodiments, the syringe contains a plunger movably inserted into the first and second chambers of the barrel. In yet further related embodiments, the plunger is configured to be fitted into each chamber of the barrel. In related embodiments, the syringe contains a first connection channel having a proximal end connected to the distal end of the first chamber of the barrel. In related embodiments, the syringe contains a second connection channel having a proximal end connected to the distal end of the second chamber of the barrel wherein the first connection channel.

In embodiments, the plunger in the syringe further contains two movably independent plungers. In related embodiments, a first plunger is inserted into a first chamber of the barrel. In related embodiments, a second plunger is inserted into a second chamber of the barrel.

In embodiments, the plunger in the syringe further contains two sections that are fixed together so as to be movably dependent. In related embodiments, a first section is inserted into a first chamber of the barrel. In related embodiments, a second section is inserted into a second chamber of the barrel.

In any of the above embodiments, the catheter is a microcatheter.

In any of the above embodiments, the catheter has at least one chamber containing a first biocompatible material. In embodiments, at least one chamber contains a second biocompatible material and/or a divalent cation to be fluidly communicated to the distal end of the catheter.

In any of the above embodiments, the catheter further contains an endoscope. In embodiments, the catheter is inserted through a lumen of an endoscope. In other embodiments, the catheter is incorporated into a design of the endoscope.

In aspects, the invention provides methods for treating a subject having a vascular or non-vascular condition. In embodiments, the methods involve providing any of the catheters described herein. In embodiments, the methods involve inserting the catheter into the subject near a targeted area. In embodiments, the methods involve administering a biocompatible material to the subject with the catheter, thereby treating the subject.

In embodiments, the vascular or non-vascular condition is any the vascular or non-vascular condition described herein, including, but not limited to, arteriovenous malformation, endovascular repair failure, osteoporosis, neurovascular lesions, telangiectasias, varicoceles, varicose veins, inflammatory lesions, hemorrhage, occlusion, embolism, neoplastic growth, venous disease, and phlebitis. In embodiments, the endovascular repair failure is endoleakage. In embodiments, the vascular occlusion is an embolism. In embodiments, the vascular occlusion is a pulmonary embolism or an arterial embolism. In embodiments, the hemorrhage is an intracranial hemorrhage.

In embodiments, the biocompatible material comprises an agent. The agent can be any agent described herein.

In related embodiments, the agent is a therapeutic agent. The therapeutic agent can be any therapeutic agent described herein.

In related embodiments, the agent is a diagnostic agent. The diagnostic agent can be any diagnostic agent described herein.

In related embodiments, the diagnostic agent is an imaging agent. The imaging agent can be any imaging agent described herein, including, but not limited to, a contrast agent, an MR imaging agent, a radio-imaging agent, an X-ray imaging agent, and a near-IR imaging agent.

In embodiments, the methods involve detecting the diagnostic agent.

In aspects, the invention provides methods for treating a subject having a neoplastic growth. In embodiments, the methods involve providing any of the catheters described herein. In embodiments, the methods involve inserting the catheter into the subject near a targeted area. In embodiments, the methods involve administering a biocompatible material to the subject with the catheter, thereby treating the subject.

In embodiments, the biocompatible material comprises an agent. The agent can be any agent described herein.

In related embodiments, the agent is an anti-cancer agent. The anti-cancer agent can be any anti-cancer agent described herein, including, but not limited to, chemotherapeutics, antibodies, and biological agents. In embodiments, the anti-cancer agent is abiraterone acetate, altretamine, anhydrovinblastine, auristatin, bexarotene, bicalutamide, BMS184476,2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine, dactinomycin, daunorubicin, dolastatin, doxorubicin, etoposide, 5-fluorouracil, finasteride, flutamide, hydroxyurea and hydroxyureataxanes, ifosfamide, liarozole, lonidamine, lomustine, mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, or vinflunine.

In embodiments, the biocompatible material further contains a diagnostic agent. In related embodiments, the diagnostic agent is an imaging agent. The imaging agent can be any imaging agent described herein, including, but not limited to, a contrast agent, an MR imaging agent, a radio-imaging agent, an X-ray imaging agent, and a near-IR imaging agent. In related embodiments, the diagnostic agent is iron oxide.

In embodiments, the methods involve detecting the diagnostic agent.

In aspects, the invention provides methods for treating or preventing osteoporosis in a subject. In embodiments, the methods involve providing any of the catheters described herein. In embodiments, the methods involve inserting the catheter into the subject near a targeted area. In embodiments, the methods involve administering a biocompatible material to the subject with the catheter, thereby treating the subject.

In embodiments, the biocompatible material comprises an agent. The agent can be any agent described herein.

In embodiments, the agent is a therapeutic agent. The therapeutic agent can be any therapeutic agent described herein. In embodiments, the therapeutic agent is an osteogenic agent. In related embodiments, the osteogenic agent is expressed by a cell. The osteogenic agent can be any osteogenic agent described herein, including, but not limited to, Wnt proteins, TGF-beta, basic fibroblast growth factor (bFGF), bone morphogenic protein-2 (BMP-2), osteonection and 1,25-dihydroxy vitamin D3 (1,25-OH D3), osteopontin, bone morphogenic proteins, Msc-2, bisphosphonates, tumor necrosis factor-alpha, oxysterols, osteoprotegerin, insulin like growth factor, high density lipoprotein, 1,25-dihydroxyvitamin D, transforming growth factor beta, estradiol, decorin, and fetuin.

In embodiments, the targeted area is osteoporotic bone. In related embodiments, the targeted area is identified by computerized tomography or magnetic resonance imaging.

In any of the above aspects and embodiments, the methods involve administering a clearing composition that dissolves the biocompatible material.

In embodiments, the clearing composition is administered to the subject with a catheter as described herein. In embodiments, the clearing composition is administered to the subject near the targeted area.

In embodiments, the clearing composition is administered to the subject systemically.

In aspects, the invention provides methods for selective dissolution of an occlusion in a subject. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering to the subject a clearing composition that dissolves the biocompatible material. In related embodiments, the clearing composition is administered near the targeted area, thereby providing selective dissolution of the occlusion in the subject.

In embodiments, the selective dissolution of the occlusion occurs in a vessel not targeted for treatment.

In embodiments, administration of the clearing composition occurs after occlusion.

In embodiments, the clearing composition is administered to the subject 1 second to 1 week after occlusion.

In aspects, the invention provides methods for selective delivery of a therapeutic agent to a targeted non-occluded vessel. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering the therapeutic agent to the subject with the catheter. In embodiments, the methods involve administering to the subject a clearing composition that dissolves the biocompatible material, thereby providing selective delivery of the therapeutic agent to the non-occluded vessel.

The therapeutic agent can be any therapeutic agent described herein. In embodiments, the therapeutic agent is a water soluble therapeutic agent.

In embodiments, administration of the clearing composition occurs after occlusion. In related embodiments, the clearing composition is administered to the subject 1 second to 1 week after occlusion.

In aspects, the invention provides methods for selective control of bulking or remodeling in a subject. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering to the subject a clearing composition that dissolves the biocompatible material, thereby providing selective control of bulking or remodeling in the subject.

In embodiments, the subject is undergoing plastic or reconstructive procedures

In embodiments, the targeted area is the lung

In aspects, the invention provides methods for lung volume reduction therapy in a subject. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering to the subject a clearing composition that dissolves the biocompatible material. In related embodiments, the clearing composition is administered near the target area, thereby providing lung volume reduction therapy in the subject.

In aspects, the invention provides methods for controlled release of a therapeutic agent in a subject. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering the therapeutic agent to the subject with the catheter. In embodiments, the methods involve administering to the subject a clearing composition that dissolves the biocompatible material, thereby providing controlled release of the therapeutic agent.

In embodiments, the biocompatible material contains the therapeutic agent.

The therapeutic agent can be any therapeutic agent described herein. In embodiments, the therapeutic agent is a water-soluble agent. In embodiments, the therapeutic agent is a chemotherapeutic agent, an anti-inflammatory agent, an antimicrobial agent, a hormonal therapy agent, a metalloproteinase inhibitor, a sclerosing agent, an angio-active agent, a plasmid for gene therapy, an adenoviral vector for gene therapy, an RNAi, an antisense molecule, a lentivirus, a microbubble, a toxin, an antibiotic, a vaccine, a photodynamic agent, and an analgesic.

In embodiments, the therapeutic agent is further combined with a second agent. The second agent can be any agent described herein, including, but not limited to, contrast agents, quantum dots, antibodies, liposomes, and nanoboxes.

In embodiments, the therapeutic agent is a cell secreting a therapeutic factor. The cell can be any cell described herein, including, but not limited to, autogenic or allogenic fibroblasts, endothelial cells, transgenic cells, mesenchymal stem cells, embryonic stem cells, extraembryonic stem cells, embryonic germ cells, cardiac stem cells, umbilical stem cells, cardiac stem cells, pluripotent and multipotent stem cells, pancreatic islet cells, hepatocytes, skin cells, intestinal stem cells, myoblasts, endothelial cells, cardiac myoblasts, dendritic cell, autologous tumor cells, monocyte derived activated killers, natural killer T cells, autologous cancer cells with liposomal IL-2, cultured chondrocytes, hematopoietic stem cells, sertoli cells, xenogenic cell sources of all listed above, skin cells, adipocytes, skin-derived stem cells, neural stem cells, glial progenitor cells, oligodendrocyte precursors, oligo precursors, fat stem cells, other stem cells sources such as from amniotic fluid, baby teeth, bone marrow cells, cord blood, placental blood, fat tissue, fetal cells, unfertilized ova, pancreas, and breast.

In embodiments, the subject has a vascular or non-vascular condition.

In embodiments, the therapeutic agent comprises a nanomaterial. In related embodiments, the therapeutic agent is contained within a nanomaterial. In related embodiments, the therapeutic agent is bound to a nanomaterial. The nanomaterial can be any nanomaterial described herein, including, but not limited to, microboxes, microchips, microfluidic pumps, magnetic resonance microcoil, quantum dots, antibody targeted nanomaterials, nanocontainers, and nanoboxes.

In embodiments, the therapeutic agent is contained within liposomes. In related embodiments, the liposome is a therapeutic liposome. In embodiments, the therapeutic liposomes are coated with protein. The protein can be any protein described herein, including, but not limited to, antibodies, receptors, and cell surface markers.

In any of the above aspects and embodiments, the biocompatible material contains tissue scaffolds, microcapsules, or wound dressings.

In aspects, the invention provides methods for the controlled release of a label in a subject. In embodiments, the methods involve providing any of the catheters described herein. In embodiments, the methods involve inserting the catheter into the subject near a targeted area. In embodiments, the methods involve administering a labeled biocompatible material to the subject with the catheter. In embodiments, the methods involve administering a clearing composition to the subject, thereby providing controlled release of the label.

In embodiments, the controlled release of the label is used for diagnostic purposes. The diagnostic purpose can be any diagnostic purpose described herein, including, but not limited to, angiography of a labeled vessel.

The label can be any label described herein, including, but not limited to, a radiolabel, a fluorescent label, and a tissue dye. The radiolabel can be any radiolabel described herein, including, but not limited to, carbon 14, carbon 14 intermediates, tritium-labeled radioisotopes, iodine 125 labeled radioisotopes, and antibody targeted radioisotopes. The fluorescent label can be any fluorescent label described herein, including, but not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be any tissue dye described herein, including, but not limited to, methylene blue.

In embodiments, the label is contained within a micelle.

In embodiments, the label is contained within a liposome.

In aspects, the invention provides methods for the controlled release of a label to mark a lesion for radiosurgery. In embodiments, the methods involve providing any of the catheters described herein. In embodiments, the methods involve inserting the catheter into the subject near a targeted area. In embodiments, the methods involve administering a labeled biocompatible material to the subject with the catheter. In embodiments, the methods involve administering a clearing composition to the subject, thereby providing controlled release of the label into and marking the lesion for radiosurgery.

The label can be any label described herein, including, but not limited to, a radiolabel, a fluorescent label, and a tissue dye. The radiolabel can be any radiolabel described herein, including, but not limited to, carbon 14, carbon 14 intermediates, tritium-labeled radioisotopes, iodine 125 labeled radioisotopes, and antibody targeted radioisotopes. The fluorescent label can be any fluorescent label described herein, including, but not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be any tissue dye described herein, including, but not limited to, methylene blue.

In embodiments, the label is contained within a micelle.

In embodiments, the label is contained within a liposome.

In aspects, the invention provides methods for controlled release of a contrast agent in a subject. In embodiments, the methods involve providing any of the catheters described herein. In embodiments, the methods involve inserting the catheter into the subject near a targeted area. In embodiments, the methods involve administering a biocompatible material to the subject with the catheter. In embodiments, the methods involve administering the contrast agent to the subject with the catheter. In embodiments, the methods involve administering to the subject a clearing composition that dissolves the biocompatible material, thereby providing controlled release of the contrast agent.

In embodiments, the biomaterial marker comprises the contrast agent.

The contrast agent can be any contrast agent described herein, including, but not limited to, magnetic resonance contrast agents, radiopaque contrast agents, ultrasound contrast agents, and nuclear medicine imaging contrast agents.

In embodiments, a portion of the biocompatible material does not dissolve when treated with the clearing composition.

In aspects, the invention provides methods for controlled release of a contrast agent in a subject. In embodiments, the methods involve providing any of the catheters described herein. In embodiments, the methods involve inserting the catheter into the subject near a targeted area. In embodiments, the methods involve administering a wound dressing comprising a biocompatible material to the subject with the catheter. In embodiments, the methods involve waiting 1 to 14 days. In embodiments, the methods involve administering a clearing composition to the subject, thereby providing selective dissolution of the wound dressing in the subject. In embodiments, the steps of administering the biocompatible material, waiting 1 to 14 days, and administering the clearing agent is repeated until the wound is healed.

In embodiments, the wound dressing comprises one or more therapeutic agents. The therapeutic agent can be any therapeutic agent described herein, including, but not limited to, analgesics, antibiotics, antifungals, antivirals, enzymes, vaccines, gene delivery vectors, antibodies, hormones, and recombinant glycoproteins.

In embodiments, the wound is a burn.

In aspects, the invention provides methods for delivering a biocompatible material to a subject. In embodiments, the methods involve providing a catheter in combination with an endoscope as described herein. In embodiments, the methods involve inserting the catheter into the subject near a targeted area. The targeted area can be any targeted area described herein, including, but not limited to, the gastrointestinal tract, the upper gastrointestinal tract, the lower gastrointestinal tract, the pulmonary tract, the larynx and the upper tracheobronchial tree, the ear, the urinary tract, the female reproductive tract, the abdominal or pelvic cavity, the interior of a joint, and an organ of the chest. In embodiments, the methods involve administering a biocompatible material to the subject with the catheter, thereby delivering the biocompatible material to the targeted area.

In aspects, the invention provides methods for delivering a biocompatible material to a subject during surgery. In embodiments, the methods involve providing a catheter in combination with an endoscope as described herein. In embodiments, the methods involve inserting the catheter into the subject near a targeted area during the surgical procedure. In embodiments, the methods involve administering a biocompatible material to the subject with the catheter, thereby delivering the biocompatible material to the targeted area during surgery.

In embodiments, the surgery is selected from the group consisting of: plastic surgery, orthopedic surgery, and endontic surgery.

In embodiments, the method further involves administering a clearing composition that dissolves the biocompatible material.

In aspects, the invention provides kit for use in treating a subject having a vascular or non-vascular condition. In embodiments, the kits contain a catheter as described herein. In embodiments, the kits further contain a biocompatible material. In embodiments, the kits further contain a clearing composition that dissolves the biocompatible material.

In aspects, the invention provides kit for use in treating a subject having a vascular or non-vascular hemorrhage. In embodiments, the kits contain a catheter as described herein. In embodiments, the kits further contain a biocompatible material. In embodiments, the kits further contain a clearing composition that dissolves the biocompatible material.

In aspects, the invention provides kit for use in treating a subject having a neoplastic growth. In embodiments, the kits contain a catheter as described herein. In embodiments, the kits further contain a biocompatible material. In embodiments, the kits further contain a clearing composition that dissolves the biocompatible material.

In aspects, the invention provides kit for the selective dissolution of an occlusion in a subject. In embodiments, the kits contain a catheter as described herein. In embodiments, the kits further contain a biocompatible material. In embodiments, the kits further contain a clearing composition that dissolves the biocompatible material.

In aspects, the invention provides kit for the selective delivery of a therapeutic agent to a targeted non-occluded vessel. In embodiments, the kits contain a catheter as described herein. In embodiments, the kits contain a biocompatible material. In embodiments, the kits contain one or more therapeutic agents. In embodiments, the kits further contain a clearing composition that dissolves the biocompatible material.

In aspects, the invention provides kit for the selective control of bulking or remodeling in a subject. In embodiments, the kits contain a catheter as described herein. In embodiments, the kits further contain a biocompatible material. In embodiments, the kits further contain a clearing composition that dissolves the biocompatible material.

In aspects, the invention provides kit for the controlled release of an agent in a subject. In embodiments, the kits contain a catheter as described herein. In embodiments, the kits contain a biocompatible material. In embodiments, the kits contain one or more agents. In embodiments, the kits further contain a clearing composition that dissolves the biocompatible material.

In aspects, the invention provides kit for the controlled release of a label in a subject. In embodiments, the kits contain a catheter as described herein. In embodiments, the kits further contain a labeled biocompatible material. In embodiments, the kits further contain a clearing composition that dissolves the biocompatible material.

In aspects, the invention provides kit for the controlled release of a label to mark lesions for radiosurgery in a subject. In embodiments, the kits contain a catheter as described herein. In embodiments, the kits further contain a labeled biocompatible material. In embodiments, the kits further contain a clearing composition that dissolves the biocompatible material.

In aspects, the invention provides kit for the controlled release of a contrast agent in a subject. In embodiments, the kits contain a catheter as described herein. In embodiments, the kits contain a biocompatible material. In embodiments, the kits contain one or more contrast agents. In embodiments, the kits further contain a clearing composition that dissolves the biocompatible material.

In aspects, the invention provides kit for the selective dissolution of a biocompatible material in a subject. In embodiments, the kits contain a catheter as described herein. In embodiments, the kits further contain a biocompatible material. In embodiments, the kits further contain a clearing composition that dissolves the biocompatible material.

In aspects, the invention provides kit for the selective dissolution of a wound dressing in a subject. In embodiments, the kits contain a catheter as described herein. In embodiments, the kits further contain a wound dressing comprising biocompatible material. In embodiments, the kits further contain a clearing composition that dissolves the biocompatible material.

In aspects, the invention provides kit for lung volume reduction therapy in a subject. In embodiments, the kits contain a catheter as described herein. In embodiments, the kits further contain a biocompatible material. In embodiments, the kits further contain a clearing composition that dissolves the biocompatible material.

In aspects, the invention provides kit for treating or preventing osteoporosis in a subject. In embodiments, the kits contain a catheter as described herein. In embodiments, the kits further contain a biocompatible material. In embodiments, the kits further comprise one or more osteogenic agents. In embodiments, the kits further contain a clearing composition that dissolves the biocompatible material.

In any of the above described aspects and embodiments, the kits can further contain a divalent cation.

In any of the above described aspects and embodiments, the kits can further contain a divalent metal chelator.

In any of the above described aspects and embodiments, the kits can further contain a instructions for use.

Unless otherwise specified, in any of the above described aspects and embodiments, the targeted area can be any area in the subject, including, but not limited to, liver, pancreas, thyroid, heart, peripheral nerve scaffold, breast, bladder, cartilage, bone, tendon, ligament, blood vessel, and spinal cord.

Unless otherwise specified, in any of the above aspects and embodiments, the biocompatible material can contain any biocompatible material described herein, including, but not limited to, a hydrazide-functionalized poly(NIPAM) co-polymer, an aldehyde-functionalized polysaccharide, a polypentapeptide elastomer, a polypeptide elastomer, an A-B-A type block copolymer elastomer, a collagen elastomer, a gelatin/chitin elastomer, a pseudopoly(amino acids) elastomer, and an alginate based biomaterial.

In embodiments, the biocompatible material can contain an aldehyde-functionalized polysaccharide selected from the group consisting of carboxymethyl cellulose (CMC), hyaluronic acid (HA), and dextran (dex).

In embodiments, the biocompatible material can contain a polypentapeptide elastomer comprising (Val-Pro-Gly-Val-Gly)ₙ.

In embodiments, the biocompatible material can contain a polypeptide elastomer comprising poly(ethylene-graft-γ-benzyl L-glutamate).

In embodiments, the biocompatible material can contain an A-B-A type block copolymer elastomer. In related embodiments, the A-B-A type block copolymer elastomer are selected from the following: i) A: poly(γ-benzyl L-glutamate) and B: polybutadiene; ii) A: poly(γ-ethyl L-glutamate) and B: polybutadiene; and iii) A: poly((γ-benzyl L-glutamate) and B: polyisoprene.

In embodiments, the biocompatible material can contain a collagen elastomer. In related embodiments, the collagen elastomer contains a collagen-polyacrylate graft copolymer.

In embodiments, the biocompatible material can contain a gelatin/chitin elastomer. In related embodiments, the gelatin/chitin elastomer contains a gelatin-carboxymethylchitin complex.

In embodiments, the biocompatible material can contain a pseudopoly(amino acids) elastomer. In related embodiments, the pseudopoly(amino acids) elastomer contains a poly(desaminotyrosyltyrosine hexylester carbonate.

In embodiments, the biocompatible material can be modified. The biocompatible material can be modified with any functional group described herein, including, but not limited to, ketone, ether, ester, amide, alcohol, amine, urea, thiourea, sulfoxide, sulfone, sulfonamide, and disulfide. In embodiments, the modification is a direct modification. In other embodiments, the modification is an indirect modification. In related embodiments, the indirect modification is through a linker molecule. In embodiments, the indirect modification involves mixing an unconjugated functional group with the biocompatible material prior to polymerization. In embodiments, the modified biocompatible material has increased binding to a charged agent as compared to an unmodified biocompatible material.

In embodiments, the biocompatible material can contain an alginate based biomaterial. The alginate based biomaterial can be any alginate based biomaterial described herein, including, but not limited to, alginic acid, D mannuronic acid and D guluronic acid, alginate, and modified alginate.

In related embodiments, the biocompatible material can contain a modified alginate. The alginate can be modified with any functional group described herein, including, but not limited to, ketone, ether, ester, amide, alcohol, amine, urea, thiourea, sulfoxide, sulfone, sulfonamide, and disulfide. In embodiments, the modification is a direct modification. In other embodiments, the modification is an indirect modification. In related embodiments, the indirect modification is through a linker molecule. In embodiments, the indirect modification involves mixing an unconjugated functional group with the alginate prior to polymerization. In embodiments, the modified alginate has increased binding to a charged agent as compared to an unmodified alginate material.

In embodiments, the modified alginate is sulfonated alginate.

The alginate can be any alginate described herein, including, but not limited to, alginate obtained from Macrocystis, Laminaria, Ascophyllum, Chlorophyceae, Phaeophyceae, Rhodophyceae, and Cyanophyceae. In embodiments, the alginate is obtained from Aminaria hyperborean. In embodiments, the alginate is obtained from Laminara digita. In embodiments, the alginate is obtained from Ascophyllum nodosum.

In embodiments, the alginate is a bacterial alginate. In related embodiments, the bacterial alginate is obtained from a heterotrophic bacteria.

Unless otherwise specified, in any of the above described aspects and embodiments, a divalent cation can be administered with the biocompatible material.

In embodiments, the divalent cation can be Ca²⁺, Mg²⁺, Ba²⁺, Sr²⁺. In embodiments, the divalent cation can be a synthetic compound with divalent orientation.

In embodiments, the divalent cation can be administered in a liposome or a microbubble. The liposome and microbubble can be any well known liposome or microbubble. In embodiments, the liposome can be a heat sensitive liposome, ultraviolet sensitive liposome, and a ph sensitive liposome.

In embodiments, the divalent cation is administered simultaneously with the biocompatible material. In other embodiments, the divalent cation is administered after administration of the biocompatible material.

Unless otherwise specified, in any of the above described aspects and embodiments, the clearing agent can be any agent capable of dissolving the biocompatible material.

In embodiments, the clearing composition contains alginate lyase.

The alginate lyase can be any alginate lyase described herein, including, but not limited to, bacterial alginate lyase. In embodiments, the alginate lyase is obtained from Flavobacterium, Burkholderia, Corynebacterium, Klebsiella, Photobacterium, Pseudoalteromonas, Pseudomonas, Rhodopirellula, Saccharophagus, Sphingomonas, Streptomyces, Vibrio, and Aspergillus. In embodiments, the alginate lyase is Flavobacterium alginate lyase. In embodiments, the alginate lyase is a transgenic alginate lyase. In embodiments, the alginate lyase, or biologically active fragment thereof, comprises SEQ ID NO: 1, or a fragment thereof.

In embodiments, the clearing composition contains a divalent metal chelator. The divalent metal chelator can be any divalent metal chelator described herein, including, but not limited to, a proteinaceous metal chelator, a non-proteinaceous metal chelator, or a calcium chelator. In embodiments, the divalent metal chelator is EDTA, DTPA, DMSA, citrate, tartrate, dimercaptol, penicillamine, deferoxamine, dithizone, cisplatin, chlorophyll, and the like.

In embodiments, the divalent metal chelator is administered together with the clearing composition.

Unless otherwise specified, in any of the above aspects and embodiments, the clearing composition is administered with the catheter.

In embodiments, the clearing composition is administered near the targeted area.

In embodiments, the clearing composition is administered systemically.

In embodiments, the divalent metal chelator and the clearing composition are both administered with the catheter.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “administration” or “administering” is meant to include an act of providing a compound or pharmaceutical composition of the invention to a subject in need of treatment.

The term “alginate” is meant to refer to the sodium salt of alginic acid. IN preferred embodiments, alginic acid refers to a linear copolymer with homopolymeric blocks of (1-4)-linked β-D-mannuronate (M) and its C-5 epimer α-L-guluronate (G) residues, respectively, covalently linked together in different sequences or blocks.

The term “alginate based biomaterial” is meant to refer to a biomaterial wherein all or a portion of the active agent contains homopolymeric blocks of (1-4)-linked β-D-mannuronate (M) and/or its C-5 epimer α-L-guluronate (G) residues. EmboGel is an example of a commercially available alginate based biomaterial that is known in the art.

The term “alginate lyase” is meant to refer to enzymes that catalyze the degradation of alginate. Alginate lyases can be characterized as either mannuronate (EC4.2.2.3) or guluronate lyases (EC 4.2.2.11), and both catalyze the degradation of alginate. Mannuronate specific alginate lyase cleaves at the β-(1-4)-D-mannuronic bonds residues of alginate to yield oligosaccharides with 4-deoxy-α-L-erythro-hex-4 enopyranuronosyl groups at their non-reducing terminus. Alginate lyases have been isolated from a wide range of organisms, including algae, marine invertebrates, and marine and terrestrial microorganisms.

The term “aneurysm” refers to the dilation, bulging, or ballooning out of part of the wall of a vein or artery. The aorta can sometimes develop an aneurysm. Aortic aneurysms usually occur in the abdomen below the kidneys. A brain aneurysm, also called a cerebral or intracranial aneurysm, is a weak bulge in the blood vessel in the brain

The terms “biocompatible material” and “biomaterial” are used interchangeably, and refer to any synthetic or natural material that can be used to replace part of a living system, or any synthetic or natural material that can function in intimate contact with living tissue.

The term “contrast agent” is meant to refer to agents that are useful in imaging techniques or methods, such as, but not limited to, magnetic resonance imaging, CT scan, ultrasound, nuclear magnetic imaging. Contrast agents can be, but are not limited to, magnetic resonance contrast agents, radiopaque contrast agents, ultrasound contrast agents, and Nuclear Medicine Imaging contrast agents.

The term “calcium agent” is meant to refer to an agent that promotes the hardening (gelation) of alginate. A calcium agent can be a solution of calcium, for example calcium chloride. A calcium agent can also refer to calcium holding containers. For example, liposomes, or microcapsules, or any other biological container that holds calcium or a calcium agent.

The term “co-administer” is intended to refer to all forms of administration that provide the alginate lyase and the divalent metal chelator, and can include sequential administration, in any order.

The term “controlled release” is meant to refer to the release of any one agent that occurs as a result of the administration of a second releasing agent. The agents can be administered in any order. In exemplary embodiments, an alginate based biomaterial comprises an agent, and an alginate lyase is used for the controlled release of the agent. For example, an alginate based biomaterial comprising an agent is administered to a subject, and a composition comprising alginate lyase and a metal chelator, for example a divalent metal chelator, is administered to the subject, thus resulting in selective release of the first agent. The selective release can be, for example, of a drug, a label, or an imaging compound.

The term “diagnosis” refers to a process of determining if an individual is afflicted with a disease or ailment, for example a vascular or non-vascular condition. A vascular condition can include arteriovenous malformation, neurovascular lesions, telangiectasias, varicoceles, varicose veins, inflammatory lesions, hemorrhage, occlusion, embolism, neoplastic growth, venous disease, and phlebitis.

The term “dissolution” or “dissolving” is meant to refer the process of breaking up or liquefying a substance into a liquid. Dissolution can mean the process of the breakdown of an alginate based biomaterial in to smaller components by an enzymatic cleavage reaction.

The term “divalent cation” or “divalent metal cation” is intended to include any metal ion with two or more possible charges. The term can also refer to a synthetic compound with appropriately spaced positive charges such that the synthetic compound has the properties of a divalent cation. Examples of divalent cations include, but are not limited to, Ca²⁺, Mg²⁺, Ba²⁺, and Sr^{2±}. In certain embodiments, the metal ion with two or more charges is contained within a liposome.

The term “divalent metal chelator” is meant to refer to a substance that binds particular ions, removing them from solution, in this case a substance that particularly removes divalent metal ions. Divalent metal chelators can be proteinaceous or non-proteinaceous chelators. Divalent metal chelators according to the invention can include, but are not limited to, EDTA, DTPA, DMSA, citrate, tartrate, dimercaptol, penicillamine, deferoxamine, dithizone, cisplatin, and chlorophyll.

The term “embolism” is meant to refer to a blockage or clot. An embolism can be the result of a blockage caused by an alginate based biomaterial. An embolism can be caused by a blood clot that travels to the lung.

The term “fiduciary marker” or “fiducial marker” refers to an object used in the field of view of an imaging system, which appears in the image produced, for use as a point of reference or a measure. It may be either something placed into or on the imaging subject, or a mark or set of marks in the reticle of an optical instrument.

The term “loaded” is meant to refer to a process of impregnating or saturating or filling another material or container. In specific embodiments, the material or container is biocompatible. For example, a biocompatible material of the invention can be loaded with alginate lyase composition.

The term “hemorrhage” is meant to refer to a discharge of blood from the blood vessels. A hemorrhage can occur in the vasculature, and is thus termed a vascular hemorrhage.

The term “nanomaterial” is meant to refer to a particle having one or more dimensions of the order of 100 nm or less. Examples of nanomaterials according to the invention include, but are not limited to, microboxes, microchips, microfluidic pumps, magnetic resonance microcoil, quantum dots, antibody targeted nanomaterials, nanocontainers, and nanoboxes.

The term “neoplastic growth” or “neoplasia” is meant to refer to any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “non-vascular condition” is meant to refer to a disease or condition that does not involve the vasculature. Non-vascular conditions are conditions that do not affect the blood vessels. Examples include, but are not limited to, a broken bone or fracture, an infection, an immunodeficiency disorder, or a metabolic disease.

The term “occlusion” or “vascular occlusion” is meant to refer to a constriction or blockage as can occur in a blood vessel. An occlusion can be the result of a blockage created with an alginate based biomaterial.

The term “simultaneously” is intended to refer to administration that occurs at the same time. The term is intended to refer to all forms of administration that provide the compositions of the invention together at the same time.

The term “subject” is intended to include vertebrates, preferably a mammal. Mammals include, but are not limited to, humans.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The term “vascular condition” is meant to refer to a condition that affects the blood vessels. Vascular conditions can include vascular disease, which affects the body's network of blood vessels (arteries and veins) that distribute oxygen and nutrient-rich blood to the body, and bring back deoxygenated blood to the heart and lungs from the rest of the body. Vascular disease can include, but is not limited to, arterial vascular disease and venous vascular disease. A vascular condition can be a vascular lesion. A vascular condition can be, but is not limited to, an occlusion, an embolism, or a hemorrhage.

The term “wound dressing” is meant to refer to a covering for a wound. The covering can be an alginate based wound covering. The alginate based covering can be a solid dressing, more specifically a solid wound dressing comprised of an alginate based biomaterial. In specific examples, the wound dressings are capable of delivering an effective wound-healing agent.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compounds, compositions, or methods provided herein can be combined with one or more of any of the other compositions and methods provided herein.

### DESCRIPTION OF THE DRAWINGS

FIG. 1 includes a graph showing the doxorubicin release profile from sulfonated glucomannan calcium alginate blend. The results demonstrate a release profile of eight days with good reproducibility between preparations in rate of release (n=10).

FIG. 2 includes a diagram image of a dual chamber syringe for delivering a two component polymerization system such as calcium chloride and alginate. By altering the relative volume of two compartments a controlled ratio of the two agents can be delivered.

FIG. 3 includes a representative catheter design to be used with the syringe showing in FIG. 2. The flexible connections allow for connection to the syringe system with rotating luer locks. Further the system includes a distal mixing tip further described in FIG. 4. A balloon channel and associated hub to attach a syringe to inflate a balloon at the distal tip may also be included in the design. Such a design can help localize delivered gel within a well defined space occluded by the balloon.

FIG. 4 includes a detailed diagram of a distal mixing tip that incorporates a side channel for calcium chloride that terminates before the distal tip. As compared to a complete concentric system, such a design reduces material and maximizes delivery space and flexibility of the catheter system.

FIG. 5 includes an Xper CT image. 1 mL of barium alginate was delivered with a concentric catheter through the working channel of an endoscope, and the image shows a well localized radiopaque mass in the lower lung field. An angiographic system (Allura Xper FD 20; Philips Medical Systems, Best, the Netherlands) was used. For each CCT acquisition, the area of interest was positioned near the system isocenter and scanned with the “propeller” movement through 240°. The scan time was 20 seconds, depending on the number of acquired images. A series of 620 images were collected at a frame rate of 30 frames per second. Each frame had a matrix size of 1024×1024 with a depth of 14 bits. The system transferred the projection image data to a workstation in parallel to the acquisition for volume data reconstruction using commercially available software (XperCT Release 1; Philips Medical Systems; Best, the Netherlands).

FIG. 6 includes a CT image. 2 mL of barium alginate was delivered to an upper lung segment, and the image shows a well localized radiopaque polymeric mass. Experiments were performed on healthy swine (40-45 kg). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

FIG. 7 includes an Xper CT comparative image of 3 mL of tantulum alginate in the upper lung segment, and 2 mL of iohexyl alginate and 1 cc of barium alginate in the lower lung segment. An angiographic system (Allura Xper FD 20; Philips Medical Systems, Best, the Netherlands) was used. For each CCT acquisition, the area of interest was positioned near the system isocenter and scanned with the “propeller” movement through 240°. The scan time was 20 seconds, depending on the number of acquired images. A series of 620 images were collected at a frame rate of 30 frames per second. Each frame had a matrix size of 1024×1024 with a depth of 14 bits. The system transferred the projection image data to a workstation in parallel to the acquisition for volume data reconstruction using commercially available software (XperCT Release 1; Philips Medical Systems; Best, the Netherlands).

FIG. 8 includes a standard fluoroscopic image. Previously delivered iohexyl alginate is adjacent to a circular fiducial marker. Below the endoscope is positioned adjacent to an area with low concentration iohexyl alginate.

FIG. 9 includes an image of a right mid-axial CT scan of 3 mL of barium alginate delivered endobronchially. Experiments were performed on healthy swine (40-45 kg). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

FIG. 10 includes an image of a right mid coronal CT scan of 3 mL of barium alginate delivered endobronchially. Experiments were performed on two healthy swine (40-45 kg). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

FIG. 11 includes an image of a right mid sagittal CT scan Experiments were performed on two healthy swine (40-45 kg). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

FIG. 12 includes a standard fluoroscopic image. Previously delivered iohexyl alginate is adjacent to a circular fiducial marker. Below the endoscope is positioned adjacent to an area with low concentration iohexyl alginate.

FIG. 13 includes CT scan images. Experiments were performed on two healthy swine (40-45 kg). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

FIG. 14 includes a graph showing the cisplatin release profile from sulfonated glucomannan calcium alginate blend.

### DETAILED DESCRIPTION OF THE INVENTION

The invention features catheters, compositions, and methods that are useful for the treatment of various conditions and diseases. The invention also provides kits and instructions for use.

The present invention is based in part on the discovery that biomaterials can be effectively delivered to a target site in a subject using a catheter as described herein.

Accordingly, the invention provides methods of treating disease and/or disorders or symptoms thereof which comprise administering a therapeutically effective amount of a pharmaceutical composition comprising a biocompatible material as described herein to a subject (e.g., a mammal such as a human). Thus, one embodiment is a method of treating a subject suffering from or susceptible to a vascular or non-vascular condition. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention in general comprise administration of a therapeutically effective amount of the compounds described herein, or a composition described herein, to a subject (e.g., animal, human) in need thereof, including a mammal, e.g., a human. Such treatment will be suitably administered to subjects, e.g., humans, suffering from, having, susceptible to, or at risk for a disease, disorder, or symptom thereof. The compounds or compositions may be also used in the treatment of any other disorders in which vascular or non-vascular lesions may be implicated.

In these aspects, the compounds and compositions are administered to the subject using a catheter as described herein. In embodiments, the catheter is used in combination with an endoscope.

As stated above, other possible delivery devices for effectively delivering biocompatible material (e.g., hydrogel) to targeted locations are ineffective. For example, the standard bronchoscope has an outer diameter of 4.9 to 5.5 mm at the distal tip and a working channel of 2.0 mm diameter and will accommodate accessories of up to 1.8 mm in diameter. Therapeutic bronchoscopes have an outer diameter of 5.8 to 6.4 mm at the distal tip and a working channel of 2.6 to 3.2 mm. At the other end of the size spectrum, bronchoscopes for pediatric indications have been made even smaller, with outer diameters between 3.5 mm down to 2.7 mm, but most of these ultra thin devices have operating channels of only 1.2 mm, which limits their suctioning and flushing capabilities and their usable accessories. Delivery of liquid embolic agents with existing devices is further complicated by the tendency for polymers to react and thus polymerize within a catheter, thereby causing catheter clogging and resistance to injection.

The present invention addresses an unmet need by providing compositions and methods that use a novel embolic forming catheter to create embolic particles of a biocompatible material (e.g., calcium alginate) that may be bland or may contain therapeutic agents.

The present invention also addresses an unmet need by providing a dual lumen catheter system with a distal mixing tip. The catheters described herein ensure that prepolymerization cannot take place as the two reacting agents are kept separate until the distal tip.

In embodiments, biomaterial (e.g., calcium alginate) is selectively delivered as a two-component polymer to a target area (e.g., airway) from microcatheters to produce effective polymer occlusion. The flow properties and the viscosity of the biomaterial (e.g., liquid alginate) can be used to optimize its delivery through microcatheters. In embodiments, a dual-lumen catheter can be used to inject the alginate and the calcium chloride reactive component simultaneously. In related embodiments, as the alginate mixes at the distal tip it polymerizes. Such catheter systems can be made of a size to fit through the lumen of an endoscope or further may be directly incorporated into the design of the endoscope.

In aspects, the catheter delivers the biomaterials described herein in combination with an endoscope.

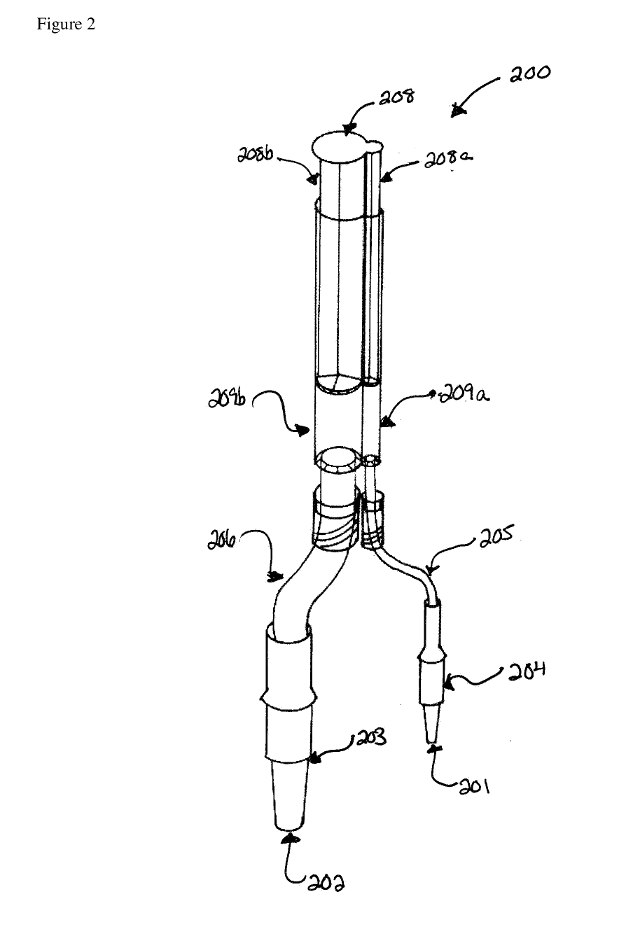

FIG. 2 an exemplary embodiment of a dual chamber syringe 200 for delivering a two component polymerization system such as calcium chloride and sodium alginate. By altering the relative volume of two compartments a controlled ratio of the two agents can be delivered. For exemplary purposes, the forgoing discussion will teach the use of calcium chloride and sodium alginate as the two delivered components forming the two component polymerization. However, it will be understood by those skilled in the art that other such components may interchangeably be applied and used without any derivation from novelty of the present invention.

Accordingly, the syringe shown in FIG. 2 includes a dual barrel 209 having a first chamber 209a and a second chamber 209b. Sealably inserted into the dual barrel 209 is a dual plunger 208 having a first section 208a corresponding with the first chamber 209a and second section 208b corresponding with the second chamber 209b. More specifically, the first chamber 209a and the second 209b are embodied so that materials contained in each chamber respectively is not allowed to come in contact with the other chamber and these materials or components (e.g., liquids) are thus prevented from being mixed until each of the respective materials reach a mixing tip (discussed below) of a catheter 300.

Although sections 208a and 208b of the dual plunger 208 are shown as being fixably attached so that they move upward and downward at the as rate and distance, it is also possible that the sections or plungers are movably independent of each other so section 208a may moved at a different rate or distance than section 208b or vice versa, thus allowing for a variable ratio of the components being applied (e.g., a variable ratio and amount of sodium alginate and calcium chloride).

The dual plunger 208 is configured to be movable in and out of the dual barrel 209 so that materials (such as sodium alginate and calcium chloride can be pushed or forced into respective delivery channels 206 and 205. A first delivery channel 205 is configure to receive, in this example, calcium chloride from the first chamber 209a of the dual barrel 209 and the second delivery channel 206 is configured to receive sodium alginate from the second chamber 209b of the dual barrel 209. The delivery channel 205 is connected on a proximal end to the first chamber 209a of the dual barrel 209 and to a connector 204 on the distal end. Likewise, the delivery channel 206 is connected to on the proximal end to the second chamber 209b of the dual barrel 209 and to a connector 206 on the distal end. The connectors 206 and 204 are configured to be connected to flexible connection portions 302 and 301 of catheter 300. Connectors 206 and 204 may for example be embodied as rotating luer locks.

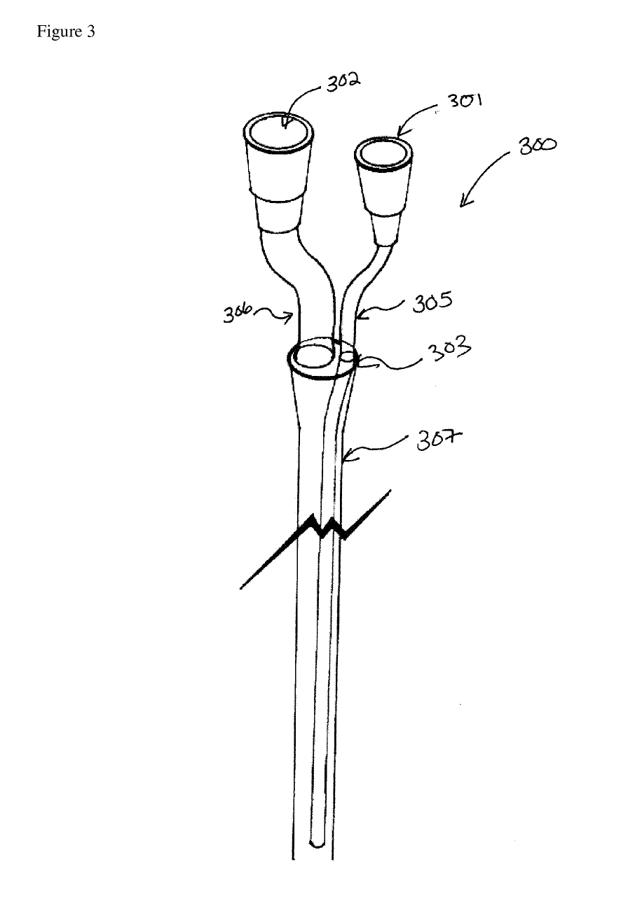

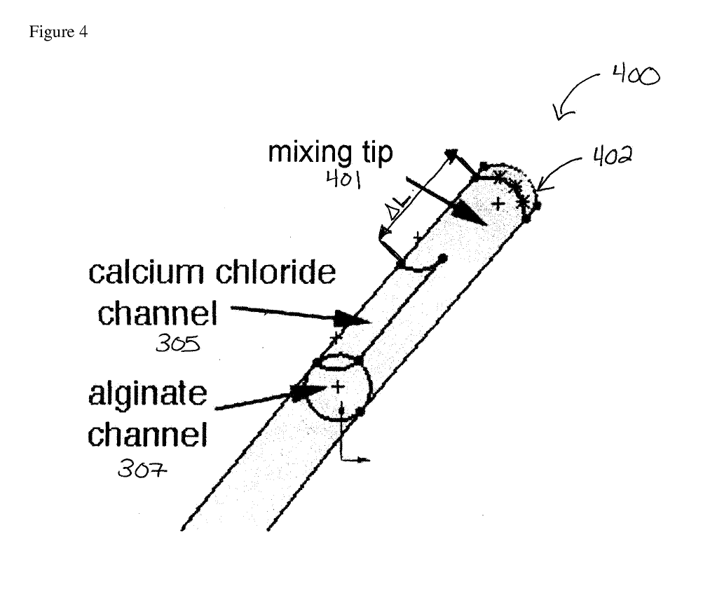

FIG. 3 includes a representative catheter design 300 to be used with the syringe 200 shown in FIG. 2. The flexible connections 301 and 302 allow for connection to the syringe system with, for example, rotating luer locks. This catheter may illustrative be a microcatheter so as to allow for use in minimally invasive procedures. Further, the catheter 300 includes a distal mixing tip 401 further described in FIG. 4. A balloon channel and associated hub to attach a syringe to inflate a balloon at the distal tip may also be included in the design. Such a design can help localize delivered gel within a well defined space occluded by the balloon.

More specifically, the catheter 300 is embodied a dual lumen catheter meaning that delivery channel 305 is disposed within or inside the delivery channel 307. Thus, the sodium alginate is delivered to the dual lumen catheter through delivery channel 306 and is then introduced into the dual lumen catheter 307. The calcium chloride, on the other hand, flows through the delivery channel 305 and is not introduced to the sodium alginate in the dual lumen catheter until it reaches the distal mixing tip 401 of the catheter 300. Thus, the delivery channel 305 is inter-disposed within the dual lumen catheter 307, preventing the sodium alginate and the calcium chloride, in this example, from being mixed until it is necessary for application.

As can be seen in FIG. 4, the delivery channel 305 is configured to be shorter in length than the end 402 of the dual lumen catheter 307. Thus, for a distance ΔL from the distal end of the catheter 307, the calcium chloride and the sodium alginate are combined thereby creating a mixing tip 401. The distance ΔL is defined as the distance from the end of the distal end of the delivery channel 305 to the distal end of the dual lumen catheter 307. This distance may be for example, about 4 inches to about 0.5 inches, where about 1 inch is preferable in most instances, depending on the diameter of the catheter. The distance, however, may be fixed or variable and is determined based upon the type of components being mixed and the required time that is required for said components to be combined properly at the distal end of the dual lumen catheter 402.

Accordingly, by providing a catheter which allows the components (e.g., the sodium alginate and calcium chloride) from being combined until they reach the distal end of a catheter, the viscosity of the fluids becomes less problematic, thereby lessening the likelihood of flow obstructions in a microcatheter. Furthermore, the amount of time required for the hydrogel to reach the targeted treatment site can be greatly decreased. Finally, by utilizing a catheter treatment sites can be more precisely targeted which in turn allows for more effective treatments.

Furthermore, in aspects of the invention, the catheter is used in combination with an endoscope. In embodiments, the catheter is inserted through a lumen of an endoscope. In other embodiments, the catheter is incorporated into a design of the endoscope.

### Biocompatible Materials

Biocompatible materials suitable for use with the above-described delivery devices are well known in the art. See, e.g., Yang et al., Chem. Soc. Rev. 40:129-137 (2011), which is hereby incorporated by reference; see also WO 2011/083086, WO 2011/082029, WO/2009/055788, WO/2009/153748, WO/2008/141059, WO/2008/124500, WO/2008/039827, WO/2007/055950, WO/2006/066234, WO/2003/105676, WO/2003/078579, WO 2007/110694, WO 2006/035444, WO 2002/065981, and WO 2002/041844, each of which is hereby incorporated by reference. Such biocompatible materials include, but are not limited to, polyvinyl alcohol, sodium polyacrylate, acrylate polymers, hyaluronase Polymers, collagen membrane, Porous HA/TCP ceramic composite, hydroxyapatite bone cement, PVP/PMMA, tricalcium phosphate, hydroxyapatite coated collagen fibers, calcium sulphate, hydroxyapatite (HAp), phosphorylcholine (PC), silicone, ultrahigh molecular weight polyethylene, polyethylene, acrylic, nylon, polyurethane, polypropylene, poly(methylmethacrylate), Teflon, Dacron, acetal, polyester, silicone-collagen composite, polyaldehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly(lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) i.e. poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites—Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers. Methods for making such materials are well known in the art.

In embodiments, the biocompatible materials are capable of forming a hydrogel, including an embolic hydrogel. In related embodiments, the hydrogel is reversible.

In embodiments, the hydrogels of the present invention are fabricated from hydrazide-functionalized poly(NIPAM) co-polymers and aldehyde-functionalized polysaccharides, such as carboxymethyl cellulose (CMC), hyaluronic acid (HA), and dextran (dex). Hydrazide-functionalized poly(NIPAM) can be generated by copolymerizing NIPAM with acrylic acid and subsequently grafting the resulting copolymer with adipic acid dihydrazide using EDC/NHS chemistry. Aldehyde-functionalized polysaccharides can be generated using periodate-mediated oxidation.

In embodiments, the polymer is a multi-component polymer system (e.g., two or three component system). See Yang et al., Chem. Soc. Rev. 40:129-137 (2011). In related embodiments, the polymer is a two component polymer system.

In embodiments, the hydrogels of the present invention comprise the elastomers provided in Table 2.

TABLE 2

| ELASTOMER | COMPOSITION |
|---|---|
| Polypentapeptide | (Val-Pro-Gly-Val-Gly)ₙ |
| Polypeptide | Poly(ethylene-graft-γ-benzyl L-glutamate) |
| A-B-A type block copolymers | A: poly(γ-benzyl L-glutamate) |
| | B: polybutadiene |
| A-B-A type block copolymers | A: poly(γ-ethyl L-glutamate) |
| | B: polybutadiene |
| A-B-A type block copolymers | A: poly ((γ-benzyl L-glutamate) |
| | B: polyisoprene |
| Collagen | Collagen-polyacrylates graft copolymer |
| Gelatin/chitin | Gelatin-carboxymethylchitin complex |
| Pseudopoly(amino acids) | Poly(desaminotyrosyltyrosine hexylester |
| | cabonate |

In aspects, the biocompatible polymer can be combined with magnetic resonance imaging and/or ultrasound contrast agents, in order to provide visibility during procedures performed with these imaging modalities. Biocompatible polymer compositions according to the invention can use magnetic resonance (MR) contrast agents such as iron-based agents, gadolinium-based agents, and fluorinated contrast agents. Specific contrast agents include bang magnetic particles, manganese oxide, gadopenteltatedimeglumine, gadoteratemeglumine (Gd-DOTA), gadodiamide injection (Gd-DTPA-BMA), gadoteridol injection (Gd-HP-D03A), gadoversetamide (Gd-DTPA-BMEA), gadobutrol (Gd-D03A-butrol), gadobenate dimeglumine (Gd-BOPTA), megafodipir trisodium (Mn-DPDP), gadoxetic acid (Gd-EOB-DTPA), feuromoxides (AMI-25), ferucarbotran (SH U 555A), gadofluorine-M, ferumoxtran (AMI-227), EP-2104R, P947, Gd-DTPA mesoporphyrin, PEG-feron (NC-100150), ferucarbotran (SH 555 C), gadofosveset (MS-325), ferumoxytol (Code 7228), gadomer-17, gadomelitol (p792), MnHa/PEG, ferric ammonium citrate, manganese chloride, manganese-loaded zeolite, ferristene (OMP), ferumoxsil (AMI-121), perfluoro-octylbromide, barium sulfate, bismuth sulfate, miscellaneous perfluorocarbons, hexafluorobenze, perfluoropolyether, Gd-DTPA, gadolinium and manganese derivatives, miscellaneous superparamagnetic iron oxide particles. In particular, bromofluorocarbons provide Hotspot imaging on 19F magnetic resonance imaging (MRI), and have sufficient radio-opacity to be conspicuous on CT, and thus are attractive agents to use.

The visibility of biocompatible polymers can be set to persist on a long-term basis, or to decrease after administration at a pace that can be controlled, for example with the use of a clearing agent. Clearing agents are known in the art and one of ordinary skill in the art would readily understand which clearing agents are suitable for use with a particular biocompatible polymer. This allows using a formulation of a biocompatible polymer that combines transient radio-opacity and long-term magnetic resonance (MR) signal. The embolic material would thus be optimally radio-opaque for safe delivery at the time of the therapeutic procedure, have its radio-opacity decrease shortly after injection in order to avoid beam-hardening artifacts on follow-up CT studies, while retaining MR signal for long-term non invasive follow up imaging studies.

Potential ultrasound agents that can be incorporated with biocompatible polymers include AI-700, Albunex, BG1135, BiSphere™, BR14, BY 963, CARDIOSPHERE, DEFINIEY, ECHOGEN, ECHOVIST-200, IMAGENT, IMAVIST, LEVOVIST, M1091, M1134, MP1950, MRX 115, MRX 408, MYOMAP, OPTISON, PESDA, Quantison, QW7437, SONAZOID, SONOGEN, SONORX, SONOVIST, SONOVUE, VISIPAQUE, ultra-small air bubbles, silica nanoparticles, perfluorocarbons, lipospheres, or any combination of shell composed of albumin, lipid, or polymer confining a gas such as nitrogen, or a perfluorocarbon.

By using liquid contrast agents as opposed to metal powders, biocompatible polymers can be safely dissolved without causing systemic release of metal powders. Radiopaque contrast agents are useful in particular embodiments of the invention. Potential radiopaque contrast agents that are useful for dissolving or combining with alginate include ethiodized oil, tantalum powder, barium sulfate, bismuth sulfate, Acetrizoic Acid Derivatives, Diatrizoic Acid Derivatives, Iothalamic Acid Derivatives, Ioxithalamic Acid Derivatives, Metrizoic Acid Derivatives, Iodamide, Lypophylic Agents, Aliphatic Acid Salts, Iodipamide, Ioglycamic Acid, Ioxaglic Acid Derivatives, Metrizamide Iopamidol, Iohexyl, Iopromide, Iobitridol, Iomeprol, Iopentol, Ioversol, Ioxilan, Iodixanol, Iotrolan, and Perfluorocarbons (PFOB).

In aspects of the invention, the biocompatible material is modified to improve binding of charged agents (e.g., therapeutic molecules). Modifications to improve the binding properties of biomaterials are well known to those of skill in the art. Such modifications include, but are not limited to, functional groups such as ketones, ethers, esters, amides, alcohols, amines, ureas, thioureas, sulfoxides, sulfones, sulfonamides, and disulfides.

In embodiments, the invention provides biomaterials that have been modified with a ketone, an ether, an ester, an amide, an alcohol, an amine, a urea, a thiourea, a sulfoxide, a sulfone, a sulfonamide, a disulfide, and the like.

The biomaterials can be modified directly or indirectly through an intervening linker. Suitable linkers are well known in the art. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, disulfide, and the like.

Additional methods for indirectly associating a functional group with a biomaterial are well known in the art. For example, in embodiments, unconjugated functional groups can be mixed with the biomaterial in the unpolymerized form and trapped within the biocompatible polymer matrix upon gelation. Agents (e.g., therapeutic molecules) would be added to the polymer blend prior to polymerization.

### Alginic Acid

In aspects of the invention, the biocompatible material is an alginate based biomaterial.

Alginate is the sodium salt of alginic acid. Sodium alginate is considered Generally Recognized as Safe (GRAS) by qualified experts, and is in accordance with United States Food and Drug Regulations. Alginic acid is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), alternating M and G-residues (MG-blocks) or randomly organized blocks. The relative amount of each block type varies both with the origin of the alginate and the concentration of G and M acids (the G/M ratio), and thus contributes to varied structural and biocompatibility characteristics. Alternating blocks form the most flexible chains, and are more soluble at lower pH than the other blocks. G-blocks form stiff chain elements, and two G-blocks of more than 6 residues each form stable cross-linked junctions with divalent cations (e.g. Ca²⁺, Mg²⁺, Ba²⁺, Sr²⁺ among others), leading to a three-dimensional gel network. Purified alginates with a high G acid content (PHG) have optimal material properties for use in endovascular occlusion. As such, alginate is a highly biocompatible material with desirable characteristics for filling and occluding vessel lesions.

Most of the alginate used commercially is obtained from three genera, Macrocystis, Laminaria, and Ascophyllum. Specific sources included Aminaria hyperborean, Laminara digita and ascophyllum nodosum. Nevertheless, alginate is present, and could potentially be isolated, from any Chlorophyceae (the green algae), Phaeophyceae (the brown algae), Rhodophyceae (the red algae) and Cyanophyceae (the blue-green algae). Alginate is also produced by two families of heterotrophic bacteria, the Pseudomonadaceae and the Azotobacteriaceae, and is often produced under strict regulatory control. The most common bacterial strains for the production of alginate are Azobacter Vinelandii and Pseudomonas Aueriginosa.

In addition, alginate can be combined with magnetic resonance imaging and/or ultrasound contrast agents, in order to provide visibility during procedures performed with these imaging modalities. Alginate compositions according to the invention can use magnetic resonance (MR) contrast agents such as iron-based agents, gadolinium-based agents, and fluorinated contrast agents. Specific contrast agents include bang magnetic particles, manganese oxide, gadopenteltatedimeglumine, gadoteratemeglumine (Gd-DOTA), gadodiamide injection (Gd-DTPA-BMA), gadoteridol injection (Gd-HP-D03A), gadoversetamide (Gd-DTPA-BMEA), gadobutrol (Gd-D03A-butrol), gadobenate dimeglumine (Gd-BOPTA), megafodipir trisodium (Mn-DPDP), gadoxetic acid (Gd-EOB-DTPA), feuromoxides (AMI-25), ferucarbotran (SH U 555A), gadofluorine-M, ferumoxtran (AMI-227), EP-2104R, P947, Gd-DTPA mesoporphyrin, PEG-feron (NC-100150), ferucarbotran (SH 555 C), gadofosveset (MS-325), ferumoxytol (Code 7228), gadomer-17, gadomelitol (p792), MnHa/PEG, ferric ammonium citrate, manganese chloride, manganese-loaded zeolite, ferristene (OMP), ferumoxsil (AMI-121), perfluoro-octylbromide, barium sulfate, bismuth sulfate, miscellaneous perfluorocarbons, hexafluorobenze, perfluoropolyether, Gd-DTPA, gadolinium and manganese derivatives, miscellaneous superparamagnetic iron oxide particles. In particular, bromofluorocarbons provide Hotspot imaging on 19F magnetic resonance imaging (MRI), and have sufficient radio-opacity to be conspicuous on CT, and thus are attractive agents to use.

The visibility of alginate can be set to persist on a long-term basis, or to decrease after administration at a pace that can be controlled, for example with the use of a clearing agent, for example alginate lyase or epimerase. This allows using a formulation of an alginate biomaterial that combines transient radio-opacity and long-term magnetic resonance (MR) signal. The embolic material would thus be optimally radio-opaque for safe delivery at the time of the therapeutic procedure, have its radio-opacity decrease shortly after injection in order to avoid beam-hardening artifacts on follow-up CT studies, while retaining MR signal for long-term non invasive follow up imaging studies.

Potential ultrasound agents that can be incorporated with alginate include AI-700, Albunex, BG1135, BiSphere™, BR14, BY 963, CARDIOSPHERE, DEFINIEY, ECHOGEN, ECHOVIST-200, IMAGENT, IMAVIST, LEVOVISTt, M1091, M1134, MP1950, MRX 115, MRX 408, MYOMAP, OPTISON, PESDA, Quantison, QW7437, SONAZOID, SONOGEN, SONORX, SONOVIST, SONOVUE, VISIPAQUE, ultra-small air bubbles, silica nanoparticles, perfluorocarbons, lipospheres, or any combination of shell composed of albumin, lipid, or polymer confining a gas such as nitrogen, or a perfluorocarbon.

By using liquid contrast agents as opposed to metal powders, alginate biomaterials can be safely dissolved without causing systemic release of metal powders. Radiopaque contrast agents are useful in particular embodiments of the invention. Potential radiopaque contrast agents that are useful for dissolving or combining with alginate include ethiodized oil, tantalum powder, barium sulfate, bismuth sulfate, Acetrizoic Acid Derivatives, Diatrizoic Acid Derivatives, Iothalamic Acid Derivatives, Ioxithalamic Acid Derivatives, Metrizoic Acid Derivatives, Iodamide, Lypophylic Agents, Aliphatic Acid Salts, Iodipamide, Ioglycamic Acid, Ioxaglic Acid Derivatives, Metrizamide Iopamidol, Iohexyl, Iopromide, Iobitridol, Iomeprol, Iopentol, Ioversol, Ioxilan, Iodixanol, Iotrolan, and Perfluorocarbons (PFOB).

Alginate can be polymerized by any divalent cation. Further, it is possible that a synthetic compound with proper divalent orientation could also replace calcium.

Alginate can be cleaved by a number of enzymes. Alginate lyases can cleave alginate. Epimerases are another class of enzymes that cleave alginate but are not specifically alginate lyases. Specifically, mannuronan c-5 epimerases, which are found in many species, can cleave alginate. The chemical mechanism and specificity of the epimerase for alginate are described by Jerga et al. (Biochemistry 2006 (45), 9138-9144), and incorporated herein by reference in its entirety.

Using alginate gels for embolization or treatment of aneurysms, including co-injection of a calcium chloride-alginate mix for polymerization has been described in WO 2005/05820, as well as U.S. Patent Application 20050133046 (Becker et al), both of which are herein incorporated by reference in their entireties. U.S. Pat. No. 6,113,629 describes radio-opaque alginate gels for the treatment of aneurysms, and is herein incorporated by reference in its entirety. Use of alginate biomaterial may include an agent for post-procedure vascular puncture closure, for filling fistulas (for example, tracheoesophageal or gastrointestinal) or surgical created fistulas, for example to fill the void where gastric tube was placed.

In aspects of the invention, the alginate is a modified alginate with improved binding of charged agents (e.g., therapeutic molecules). Modifications to improve the binding properties of alginate are well known to those of skill in the art. Such modifications include, but are not limited to, functional groups such as ketones, ethers, esters, amides, alcohols, amines, ureas, thioureas, sulfoxides, sulfones, sulfonamides, and disulfides.

In embodiments, the invention provides alginates that have been modified with a ketone, an ether, an ester, an amide, an alcohol, an amine, a urea, a thiourea, a sulfoxide, a sulfone, a sulfonamide, a disulfide, and the like. In embodiments, the invention relates to the use of sulfonated alginate.

Alginate can be modified directly or indirectly through an intervening linker Suitable linkers are well known in the art. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, disulfide, and the like.

Additional methods for indirectly associating a functional group with alginate are well known in the art. For example, in embodiments, unconjugated functional groups can be mixed with alginate in the unpolymerized form and trapped within the alginate polymer matrix upon gelation of alginate. Agents (e.g., therapeutic molecules) would be added to the polymer blend prior to polymerization.

### Alginate Lyase

Alginate lyases, characterized as either mannuronate (EC 4.2.2.3) or guluronate lyases (EC 4.2.2.11) catalyze the degradation of alginate, a complex copolymer of α-L-guluronate and its C5 epimer β-D-mannuronate. Alginate lyase cleaves at the b-(1-4)-D-mannuronic bonds residues of alginate to yield oligosaccharides with 4-deoxy-a-L-erythro-hex-4 enopyranuronosyl groups at their non-reducing terminus Alginate enzymatic hydrolysis with the alginate lyase enzyme creates polymannuronic acid (MW 5-10 kD). Alginate lyases have been isolated from a wide range of organisms, including algae, marine invertebrates, and marine and terrestrial microorganisms (Wong, T Y et al. Ann Rev of Microbiol 2000 54: 289-340, herein incorporated by reference).

Alginate lyase can be obtained from a number of sources; including bacterial sources. The production of alginate lyase from Enterobacter cloacae is described in U.S. Pat. No. 5,348,875, which is herein incorporated by reference in its entirety. Table 1 below lists exemplary sources of alginate lyase:

TABLE 1

| Protein | Organism |
|---|---|
| Bcep18194_B2401 | Burkholderia sp. 383 |
| guluronate lyase (alyPG) | Corynebacterium sp. ALY-1 |
| alginate lyase (AlyA) | Klebsiella pneumoniae subsp. |
| | aerogenes |
| alginate lyase AlxM | Photobacterium sp. ATCC 43367 |
| Patl_3645 | Pseudoalteromonas atlantica T6c |
| Patl_3639 | Pseudoalteromonas atlantica T6c |
| PSHAa0571 | Pseudoalteromonas haloplanktis |
| | TAC125 |
| alginate lyase (PA1167) | Pseudomonas aeruginosa PAO1 |
| PA1784 | Pseudomonas aeruginosa PAO1 |
| PFL_5780 | Pseudomonas fluorescens Pf-5 |
| PFL_3421 | Pseudomonas fluorescens Pf-5 |
| PFL_4740 | Pseudomonas fluorescens Pf-5 |
| Pfl_5256 | Pseudomonas fluorescens PfO-1 |
| PP3774 | Pseudomonas putida KT2440 |
| PSPPH_3296 | Pseudomonas syringae pv. |
| | phaseolicola 1448A; BAA-978 |
| PSPPH_0498 | Pseudomonas syringae pv. |
| | phaseolicola 1448A; BAA-978 |
| Psyr_0508 | Pseudomonas syringae pv. syringae |
| | B728a |
| Psyr_3376 | Pseudomonas syringae pv. syringae |
| | B728a |
| PSPTO5015 | Pseudomonas syringae pv. tomato str. |
| | DC3000 |
| PSPTO3605 | Pseudomonas syringae pv. tomato str. |
| | DC3000 |
| RB3601 | Rhodopirellula baltica SH 1 |
| Sde_1507 | Saccharophagus degradans 2-40 |
| Sde_3286 | Saccharophagus degradans 2-40 |
| Sde_2839 | Saccharophagus degradans 2-40 |
| Sde_2478 | Saccharophagus degradans 2-40 |
| Sde_2873 | Saccharophagus degradans 2-40 |
| Sde_2547 | Saccharophagus degradans 2-40 |
| alginate lyase (A1-II′) | Sphingomonas sp. A1 |
| alginate lyase (aly; A1-I/PolyG + | Sphingomonas sp. A1 |
| PolyM; A1-II/PolyG; A1-III/PolyM) | |
| SAV802 | Streptomyces avermitilis MA-4680 |
| Q9RKE1 or SCE65.33c | Streptomyces coelicolor A3(2) |
| alginate lyase (Aly1) (fragment) | Streptomyces sp. MET0515 |
| alginate lyase AlyVGII | Vibrio halioticoli IAM14596T |
| alginate lyase AlyVGI | Vibrio halioticoli IAM14596T |
| alginate lyase (AlyVI) | Vibrio sp. QY101 |
| AO090020000698 | Aspergillus oryzae RIB 40 |

An exemplary alginate lyase according to the invention, alginate lyase from Pseudomonas aeruginosa, is shown in SEQ ID NO: 1.

SEQ ID NO: 1
MKTSHLIRIA LPGALAAALL ASQVSQAADL VPPPGYYAAV GERKGSAGSC PAVPPPYTGS
LVFTSKYEGS DSARATLNVK AEKTFRSQIK DITDMERGAT KLVTQYMRSG RDGDLACALN
WMSAWARAGA LQSDDFNHTG KSMRKWALGS LSGAYMRLKF SSSRPLAAHA EQSREIEDWF
ARLGTQVVRD WSGLPLKKIN NHSYWAAWSV MSTAVVTNRR DLFDWAVSEF KVAANQVDEQ
GFLPNELKRR QRALAYHNYA LPPLAMIAPF AQVNGVDLRQ ENHGALQRLA ERVMKGVDDE
ETFEEKTGED QDMTDLKVDN KYAWLEPYCA LYRCEPNACS RPKKDREPFN SFRLGGEVTR
VFSREGGS

An exemplary source of alginate lyase according to the invention is isolated from Flavobacterium. In certain embodiments, modification to alginate lyase may be made to enhance activity and/or reduce toxicity. Exemplary modifications include pegylation or chemical modification. Other modifications to alginate lyase are made to increase the speed of dissolution of alginate. Some modifications include, but are not limited to, the addition of buffering agents, including glycine; sodium citrate; citric acid, bicarbonate buffers (sodium carbonate, sodium bicarbonate), phosphate buffer, protein buffer, TRIS (tromethamine) buffer, veronal buffer, Krebs buffer, Butterfield phosphate buffer, lactic acid, 3-{[tris(hydroxymethyl)methyl]amino}propanesulfonic acid, N,N-bis(2-hydroxyethyl)glycine, tris(hydroxymethyl)methylamine, N-tris(hydroxymethyl)methylglycine, 4-2-hydroxyethyl-1-piperazineethanesulfonic acid, 2-{[tris(hydroxymethyl)methyl]amino}ethanesulfonic acid, 3-(N-morpholino) propanesulfonic acid, piperazine-N,N′-bis(2-ethanesulfonic acid), dimethyl arsenate, 2-(N-morpholino)ethanesulfonic acid, acetate, citric acid-phosphate buffer, MES, ADA, PIPES (piperazine-N,N′-bis(2-ethanesulfonic acid)), ACES (N-(2-Acetamido)-2-aminoethanesulfonic acid), Cholamine chloride, BES, TES, Acetamidoglycine, Tricine, Glycinamide, Bicine, [and substitution of other chelating agents, such as DTPA or DMSA in place of EDTA.

The use of alginate lyase compositions of the invention is not limited to dissolving alginate in strand form only. The compositions can also be use to dissolve or partially dissolve materials that consist entirely or in part of alginate. Such materials include but are not limited to tissue scaffolds, microcapsules, caps or spheres, and wound dressings. In particular embodiments, alginate lyase compositions may prove highly useful in the release of drug or radioisotope-containing liposomes from alginate microcapsules to a targeted tissue. A target organ or tissue could be embolized with alginate microcapsules containing therapeutic liposomes, which could then be released by systemic or selective administration of alginate lyase, or any enzyme that dissolves alginate.

Alginate lyase on its own has therapeutic potential. Studies looking at the safety and efficacy of bacterial alginate lyase are already underway in animal models, in regard to the potential use of alginate lyase in the treatment of cystic fibrosis patients infected with Pseudomonas aeruginosa, which proliferates in alginate biofilms.

### EmboCaps

Alginate based biomaterials can form EmboCaps. EmboCaps are in a small spherical form, and are polymerized prior to injecting in the body. In embodiments, EmboCaps are used with intravascular delivery strategies, as microcapsules can be used as embolic agents to create a reversible stasis thereby allowing a high payload of therapeutic agent to be delivered to a relatively well-targeted area. In embodiments, EmboCaps are used as transport vectors for the delivery and/or controlled release of a large array of bioactive agents, including, but not limited to, chemotherapeutics, anti-inflammatories, antimicrobials, hormonal therapy agents, gene therapy vectors, or radioisotopes for radiotherapy.

The rate of diffusion of bioactive agent from alginate capsules can be altered by modifying the porosity of the matrix. Alginate can be readily coated with a rate-controlling, size-selective membrane of cationic polypeptides such as poly-1-lysine and poly-1-ornithine. The properties of the coating can be controlled by varying the parameters of the coating process such as the coating material, its molecular weight, the concentration of the coating solution, and the coating time, allowing the design of coatings with different molecular weight cut offs and with different release rates. In addition to diffusion controlled-strategies the release of bioactive agent from hydrogels can be erosion-controlled. By adding biodegradable components to the hydrogel such as collagen or hyaluronic acid, the rate of drug release can be determined by the rate of erosion of the biodegradable agent.

EmboCaps consist of an alginate matrix with controllable porosity that provides a means of diffusion-controlled release of a therapeutic agent. EmboCaps can also utilize erosion controlled release with the addition of EmboClear, an alginate dissolving solution that has been shown to have minimal toxicity in vivo. By adjusting the porosity of EmboCaps and varying the delivery time and dosage of EmboClear after embolization, the clinician is given unprecedented control over the release of bioactive agents from an embolic particle.

### Methods of the Invention

The present invention provides methods for administering to a subject a biocompatible material (e.g., hydrogel) using a catheter as described herein. In embodiments, the biocompatible material is an embolic hydrogel comprising a polymer (e.g., alginate polymer) suitable for delivery to a targeted site in the subject (e.g., transbronchial delivery site). In related embodiments, the hydrogel comprises a multi-component polymer system (e.g., two or three component system). In embodiments, the hydrogel further comprises an agent (e.g., therapeutic agent, fiduciary marker, and the like) for delivery to the targeted site in the subject.

In aspects, the methods involve using a catheter as described herein in combination with an endoscope. An endoscope is a medical instrument used to examine the interior cavity of a subject. An endoscope can comprise any combination of the following components: a rigid or flexible tube, a light delivery system to illuminate the organ or object under inspection, a lens system transmitting the image to the viewer from the objective lens to the viewer, an eyepiece, and an additional channel to allow entry of medical instruments or manipulators (e.g., a lumen). Endoscopic applications are well known in the art. For example, endoscopes are suitable for use to examine the gastrointestinal tract (esophagogastroduodenoscopy, esophagoscopy, enteroscopy, colonoscopy, sigmoidoscopy, magnification endoscopy, endoscopic retrograde cholangiopancreatography, endoscopic retrograde cholangiopancreatography, duodenoscope-assisted cholangiopancreatoscopy, intraoperative cholangioscopy, rectoscopy, anoscopy, proctoscopy, and the like), the upper gastrointestinal tract (panendoscopy), the larynx and the upper tracheobronchial tree (laryngoscopy), the pulmonary/respiratory tract (rhinoscopy, bronchoscopy, and the like), the ear (otoscopy and the like), the urinary tract (cystoscopy and the like), the female reproductive tract (gynoscopy, colposcopy, hysteroscopy, falloposcopy, and the like), the abdominal or pelvic cavity (laparoscopy and the like), the interior of a joint (arthroscopy and the like), the organs of the chest (thoracoscopy, mediastinoscopy, and the like), and the like. Endoscopes are also suitable for use during plastic surgery, orthopedic surgery, endodontic surgery, and the like.

Thus, in embodiments, a catheter as described herein is used in combination with an endoscope to administer a biocompatible material as described herein to the gastrointestinal tract, the upper gastrointestinal tract, the lower gastrointestinal tract, the pulmonary/respiratory tract, the larynx and the upper tracheobronchial tree, the ear, the urinary tract, the female reproductive tract, the abdominal or pelvic cavity, the interior of a joint, the organs of the chest, and the like. In embodiments, a catheter as described herein is used in combination with an endoscope to administer a biocompatible material during plastic surgery, orthopedic surgery, endodontic surgery, and the like.

In aspects, the invention relates to catheters, compositions, and methods for forming embolic material in order to provide occlusion of pulmonary airways to treat conditions that require an embolization to alleviate the condition (e.g., fistula) and targeted delivery of therapeutic agents. In embodiments, the invention provides catheters, compositions, and methods that use hydrogels to fill pulmonary airways. As described in detail herein, such hydrogels include any suitable biomaterial known in the art, including, but not limited to, calcium alginate. Suitable catheters are described herein, and include but are not limited to, a concentric catheter capable of delivering the hydrogel to the target site.

In aspects, the methods comprise administering to the subject a composition comprising a biomaterial capable of forming a hydrogel. In embodiments, hydrogel formation is reversible. The biocompatible material may be any biocompatible material well known in the art, including, but not limited to, those biomaterials described herein (e.g., alginate).

In related embodiments, the biomaterial is a polymer selected from the group consisting of polypentapeptide elastomers (e.g., (Val-Pro-Gly-Val-Gly)ₙ); polypeptide elastomers (e.g., poly(ethylene-graft-γ-benzyl L-glutamate); A-B-A type block copolymers such as A: poly(γ-benzyl L-glutamate)-B: polybutadiene copolymers, A: poly(γ-ethyl L-glutamate)-B: polybutadiene polymers, and A: poly((γ-benzyl L-glutamate)-B: polyisoprene; collagen elastomers such as collagen-polyacrylates graft copolymer; gelatin/chitin elastomers such as gelatin-carboxymethylchitin complexes; and pseudopoly(amino acids) elastomers such as poly(desaminotyrosyltyrosine hexylester carbonate polymers.

In embodiments, the polymer is a multi-component polymer system. In related embodiments, the polymer is a two or three component polymer system.

In embodiments, the biomaterial is an alginate polymer. In related embodiments, the alginate polymer is a multi-component system, including a two or three component system (e.g., calcium alginate system described in detail herein).

In embodiments, the methods involve administering to the subject a composition comprising an alginate lyase and a divalent metal chelator. In related embodiments, the compositions may or may not comprise a divalent metal chelator. In certain embodiments, the methods of the invention use the alginate lyase composition without a divalent metal chelator to slow down the dissolution of the alginate based biomaterial in a subject, or in embodiments where a divalent metal chelator, such as EDTA, might be toxic to an individual. For instance, application of the alginate lyase composition without a divalent metal chelator may be appropriate in situations where the alginate based biomaterial is used as a wound dressing, e.g. as smartskin, as described herein.

Alginate based biomaterials can be selectively dissolved after application, using an alginate lyase based composition. The final product of the dissolution consists of a biocompatible molecule. This property adds safety to endovascular procedures, since the passage of embolic material in a non-targeted vessel, a complication with potentially devastating consequences, can be rapidly reversed by selective dissolution with alginate lyase. Reported instances of such untoward events include for example migration of embolic agent into a brain artery causing a stroke, or into a pulmonary artery causing a pulmonary embolism. In addition to this safety features, the dissolving property of alginate lyase can be used for selective release of bioactive agents, in remote locations and at a controlled pace.

In embodiments, the alginate based biomaterial is Embogel. Embogel is an alginate composition with controllable porosity that provides diffusion or erosion-controlled release of therapeutic agents. Embogel permits encapsulation of liposomes containing small molecule therapeutics for extended drug release profiles; is suitable for incorporation of contrast agents, allowing for visual assessment of microsphere location with MRI, CT, or ultrasound; is non-adhesive to reduce the risk of microcatheter tip retention; and is radio-opaque.

In aspects, the invention provides methods for delivering bland particles, e.g., those that do not contain any toxic agent, so non-targeted particles can be dissolved without the non-targeted release of agent. Alternatively if a toxic agent is used, it is safer if the agent becomes activated in some way by, for example, ultraviolet (UV) light, or ultrasound (US) rupture. In the event of non-targeted delivery, the biocompatible (e.g., alginate) capsules can be lysed, and the non-activated agent can clear the system before activation of the targeted agent.

In aspects, the present invention provides methods for selectively delivering an agent (e.g., therapeutic agent) to a targeted vessel/site. Delivery of the agent is achieved in a highly selective manner through the use a catheter as described herein. The catheter delivers the biocompatible material (e.g., alginate based biomaterial) so that vessels occlude in the area where the agent is not desirably delivered, and leaving non-occluded vessels free for agent delivery in the area of treatment. Further, a composition to dissolve the biomaterial (e.g., alginate lyase), can be used at the end of treatment, to dissolve the occluded vessel.

In aspects, the invention provides methods to selectively deliver an agent (e.g., a therapeutic agent) to a targeted non-occluded vessel. In embodiments, the methods involve administering a biomaterial (e.g., alginate based biomaterial) comprising the agent to the targeted non-occluded vessel. In related embodiments, administration of a composition that dissolves the biomaterial (e.g., alginate lyase) to the targeted area provides selective delivery of the therapeutic agent to the non-occluded vessel. Optionally, in embodiments where alginate lyase is administered to the subject, the alginate lyase is administered in combination with a divalent metal chelator. In embodiments, the subject has previously received treatment with a biomaterial.

In embodiments, the methods further involve administering to the subject a composition that dissolves the biomaterial (e.g., alginate lyase composition) after occlusion. In related embodiments, the composition is alginate lyase in combination with a divalent metal chelator.

In embodiments, administering a composition (e.g., alginate lyase) that dissolves the occlusion or dissolves the biomaterial occurs any time after occlusion or administration of the biomaterial, for example 5 minutes, 15 minutes, 30 minutes, 1 hour, 2 hours, 5 hours, 10 hours, 24 hours, or more.

Water-soluble drugs can easily be dissolved in the biocompatible material (e.g., alginate) and become trapped in the resulting matrix once the sample is gelled, allowing for drug-enhanced embolization. This characteristic can be further enhanced by using a biomaterial/hydrogel (e.g., alginate gels) having a porous structure that allows for controlled drug diffusion. In addition, the present methods can use biomaterials that can be selectively dissolved (e.g., alginate gels can be dissolved with an alginate lyase composition). Thus, if the embolic biomaterial is delivered in a non-targeted structure, for example a blood vessel feeding normal tissue, it can be broken down into biocompatible liquid components again.

Any agent can be delivered in this manner. Exemplary agents include, but are not limited to, chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins (ricin toxin, conotoxin, botulin toxin a-g, diptheria toxin, cholera toxin, tetanus toxin, shiga-like toxin antibiotics, vaccines, photodynamic agents, alpha emitters, beta emitters, antibodies, hormones, recombinant glycoproteins and analgesics.

In embodiments, the invention provides methods for selective dissolution of a biocompatible material, wherein the material consists only in part of alginate and therefore partially dissolves when treated with alginate lyase. In embodiments, the methods involve administering to the subject an alginate loaded biocompatible material to a targeted area. In embodiments, the methods involve administering to the subject a composition comprising alginate lyase to the targeted area of the first step. In related embodiments, administration of the composition comprising alginate lyase provides selective dissolution of the biocompatible material in the subject. In certain embodiments, the composition may further comprise a divalent metal chelator.

The targeted area according to the method of the invention is any area that is in need of a biocompatible material. The targeted area could be a target organ in need of treatment, including, but not limited to, an area requiring a composite for artificial muscle, artificial hearts and pacemakers, tissue-engineered human heart tissue, artificial pancreas, artificial liver, artificial blood vessel, artificial nerves, drug/gene delivery stent, nerve graft and the like. In embodiments, the targeted area is selected from the group consisting of: liver, pancreas, thyroid, heart, peripheral nerve scaffold, breast, bladder, cartilage, bone, tendon, ligament, blood vessel, spinal cord, and the like.

In embodiments, the biomaterial (e.g., alginate) can be incorporated in combination with any material that is transplantable in to the human body. In embodiments, the biomaterial is a component of the catheter described herein. In embodiments, the biomaterial, for example, may be a component of a polymer based stent or an artificial valve. Administration of a dissolving composition (e.g., alginate lyase) could cause partial breakdown of the catheter, resulting in the release of an agent (e.g., therapeutic agent, including genetic material). The additional biocompatible material can be, but is not limited to: polyvinyl alcohol, sodium polyacrylate, acrylate polymers, hyaluronase Polymers, collagen membrane, porous HA/TCP ceramic composite, hydroxyapatite bone cement, PVP/PMMA, tricalcium phosphate, hydroxyapatite coated collagen fibers, calcium sulphate, hydroxyapatite (HAp), phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly(lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids), poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, methylacrylate, para-xylylene, biodegradable copolymers, copolymer surface coatings, starch polymers, polylactic acid, cellophane, tyrosine polycarbonates, lactide and glycolide polymers, collagen, PTFE, silicone, keratin-based materials, fibrous composites—carbon fiber and particles, polymer composites, artificial/natural material composites, glass-ceramic/metal composites, glass-ceramic/nonmetal composites, dental composites, Ormocer, hydrogels, timed-release foams, and polymeric carriers.

Vascular and Non-Vascular Conditions

The compositions of the present invention are useful for a variety of bioapplications (e.g., hydrogels comprising a biocompatible material), such as occluding blood vessels, occluding aneurysms, occluding other body lumens such as fallopian tubes, filling aneurysm sacs, as arterial sealants, as puncture sealants, and the like. The compositions are delivered to a targeted site in a subject using a catheter as described herein.

In aspects, the invention provides for treating a subject suffering from a vascular or non-vascular condition. In embodiments, the method involves the step of administering to the subject a composition comprising a biomaterial as described herein (e.g., alginate polymer). In related embodiments, the method involves the step of administering a compound that reverses biomaterial polymerization (e.g., an alginate lyase, optionally a divalent metal chelator, and the like). In embodiments, the subject has previously received treatment with a biocompatible composition (e.g., alginate hydrogel).

In aspects, the invention provides for treating a subject suffering from a vascular or non-vascular occlusion. In embodiments, the method involves the step of administering to the subject a composition comprising a biomaterial as described herein (e.g., alginate polymer). In related embodiments, the method involves the step of administering a compound that reverses biomaterial polymerization (e.g., an alginate lyase, optionally a divalent metal chelator, and the like). In embodiments, the subject has previously received treatment with a biocompatible composition (e.g., alginate hydrogel).

In aspects, the invention provides for treating a subject suffering from a vascular or non-vascular hemorrhage. In embodiments, the method involves the step of administering to the subject a composition comprising a biomaterial as described herein (e.g., alginate polymer). In related embodiments, the method involves the step of administering a compound that reverses biomaterial polymerization (e.g., an alginate lyase, optionally a divalent metal chelator, and the like). In embodiments, the subject has previously received treatment with a biocompatible composition (e.g., alginate hydrogel).

A “vascular condition” is a condition that affects the blood vessels. Non-vascular conditions are conditions that do not affect the blood vessels.

Vascular conditions include vascular diseases that affect the body's network of blood vessels (arteries and veins) that distribute oxygen and nutrient-rich blood to the body, and bring back deoxygenated blood to the heart and lungs from the rest of the body. Arterial vascular disease is primarily caused by fatty deposits called plaque that lead to hardening of the arteries, or atherosclerosis. This can restrict blood flow in areas outside the heart, including the legs, arms, brains, torso and neck. (The term “cardiovascular” refers to the heart and its network of arteries and veins.) Arterial vascular disease includes stroke, aneurysms, carotid artery disease, varicose veins and more.

Venous vascular disease primarily affects the veins in the legs, caused by plaque build-up that blocks blood flow or stagnant blood flow or injury to blood vessels.

A vascular condition can be a vascular lesion. Arteriovenous malformations (AVMs) are defects of the circulatory system that are generally believed to arise during embryonic or fetal development or soon after birth. They are comprised of snarled tangles of arteries and veins. Arteries carry oxygen-rich blood away from the heart to the body's cells; veins return oxygen-depleted blood to the lungs and heart. The presence of an AVM disrupts this vital cyclical process. Although AVMs can develop in many different sites, those located in the brain or spinal cord can have especially widespread effects on the body. One of the greatest potential dangers posed by AVMs is hemorrhage. Information on AVMs can be found on the world wide web at ninds.nih gov/disorders/avms/detail_avms.htm.

AVMs can form virtually anywhere in the brain or spinal cord—wherever arteries and veins exist. Some are formed from blood vessels located in the dura mater or in the pia mater, the outermost and innermost, respectively, of the three membranes surrounding the brain and spinal cord. (The third membrane, called the arachnoid, lacks blood vessels.) AVMs affecting the spinal cord are of two types, AVMs of the dura mater, which affect the function of the spinal cord by transmitting excess pressure to the venous system of the spinal cord, and AVMs of the spinal cord itself, which affect the function of the spinal cord by hemorrhage, by reducing blood flow to the spinal cord, or by causing excess venous pressure. Spinal AVMs frequently cause attacks of sudden, severe back pain, often concentrated at the roots of nerve fibers where they exit the vertebrae; the pain is similar to that caused by a slipped disk. Dural and pial AVMs can appear anywhere on the surface of the brain. Those located on the surface of the cerebral hemispheres—the uppermost portions of the brain—exert pressure on the cerebral cortex, the brain's “gray matter.” Depending on their location, these AVMs may damage portions of the cerebral cortex involved with thinking, speaking, understanding language, hearing, taste, touch, or initiating and controlling voluntary movements. AVMs located on the frontal lobe close to the optic nerve or on the occipital lobe, the rear portion of the cerebrum where images are processed, may cause a variety of visual disturbances. AVMs also can form from blood vessels located deep inside the interior of the cerebrum. These AVMs may compromise the functions of three vital structures: the thalamus, which transmits nerve signals between the spinal cord and upper regions of the brain; the basal ganglia surrounding the thalamus, which coordinate complex movements; and the hippocampus, which plays a major role in memory. AVMs can affect other parts of the brain besides the cerebrum, including the hindbrain and the brainstem.

Besides AVMs, three other main types of vascular lesion can arise in the brain or spinal cord: cavernous malformations, capillary telangiectases, and venous malformations. These lesions may form virtually anywhere within the central nervous system, but unlike AVMs, they are not caused by high-velocity blood flow from arteries into veins. In contrast, cavernous malformations, telangiectases, and venous malformations are all low-flow lesions. Instead of a combination of arteries and veins, each one involves only one type of blood vessel. These lesions are less unstable than AVMs and do not pose the same relatively high risk of significant hemorrhage.

Thus the methods of the invention can be used to treat AVMs, including AVMs located deep inside the brain. For example, in endovascular embolization the surgeon guides a catheter though the arterial network until the tip reaches the site of the AVM. The surgeon then introduces a substance that will plug the fistula, correcting the abnormal pattern of blood flow. This process is known as embolization because it causes an embolus (a blood clot) to travel through blood vessels, eventually becoming lodged in a vessel and obstructing blood flow. The materials used to create an artificial blood clot in the center of an AVM include fast-drying biologically inert glues, fibered titanium coils, and tiny balloons. In exemplary embodiments, the compositions and methods of the invention are suited for use in the method, either alone, or as an adjunct to surgery or to radiosurgery to reduce the blood flow through the AVM and make the surgery safer.

Also treated by the methods of the invention are vascular conditions such as varicose veins. Varicose veins are swollen and twisted veins that are visible just under the surface of the skin. They appear most commonly in the legs, but also can develop in other parts of the body. A number of other types of vein problems are related to varicose veins, for example telangiectasias are small clusters of blood vessels that look similar to spider veins. They are red in color and are commonly found on the upper body, including the face. They can develop during pregnancy and in people who have certain genetic disorders, viral infections, and other medical conditions (such as liver disease). The methods of the invention can be used, for example, to ablate the damaged varicose vein.

The methods of the invention can be used to treat hemorrhage in a subject. Hemorrhage is the medical term for bleeding, and means escape of blood to extravascular space. An intracerebral hemorrhage is bleeding in the brain caused by the rupture of a blood vessel within the head. Internal bleeding can occur in any part of the brain. Bleeding in the brain irritates the brain tissues, causing swelling (cerebral edema). The blood may collect into a mass (hematoma). Both cerebral edema and the presence of a hematoma within the brain put increasing pressure on the brain tissues and eventually destroy them. Deep intracerebral hemorrhage is a type of stroke caused by bleeding within the deep structures of the brain (thalamus, basal ganglia, pons, and cerebellum). Lobar intracerebral hemorrhage is bleeding in the largest part of the brain called the cerebrum. Lobar intracerebral hemorrhage (ICH) may be caused by traumatic brain injury or blood vessel problems, such as aneurysm, arteriovenous malformation, or angioma, a type of blood vessel tumor.

Also treated by the methods of the invention are vascular occlusions. A vascular occlusion is blockage of a blood vessel. In embodiments, the blockage of a blood vessel is treated with the compositions and apparatuses described herein. In related embodiments, the vascular occlusion is an embolism. An embolism can be a pulmonary embolism or an arterial embolism. A pulmonary embolism is a sudden blockage in a lung artery. In general, a pulmonary embolism is usually due to a blood clot that traveled to the lung from the leg. A clot that forms in one part of the body and travels in the bloodstream to another part of the body is called an embolus. The type of clot that is likely to cause a pulmonary embolism originates in the veins deep in your muscles. This condition is called deep vein thrombosis (DVT). DVT usually occurs in your leg or pelvis veins, although less commonly it can also sometimes occur in your arm veins. Arterial embolism is a sudden interruption of blood flow to an organ or body part due to a clot (embolus). Arterial emboli often occur in the legs and feet. Some may occur in the brain, causing a stroke, or the heart, causing a heart attack. Less common sites include the kidneys, intestines, and the eyes.

In aspects of the invention, emboli are due to the vascular migration of the biomaterials.

In embodiments, vascular or non-vascular conditions are selected from the group consisting of: arteriovenous malformation, neurovascular lesions, telangiectasias, varicose veins, inflammatory lesions, hemorrhage, occlusion, embolism, neoplastic growth, venous disease, and phlebitis.

In embodiments, the invention provides methods to treat vascular leaks, for example, endoleaks. Vascular leakage, e.g., endoleak, is a major complication and its persistence following endovascular aortic aneurysm repair indicates a failure of the procedure. Its detection and treatment is therefore of primary importance, since endoleak can be associated with pressurization (increase in pressure) of the sac, resulting in expansion and rupture of the aneurysm. A biocompatible liquid embolic agent offers some degree of control and is of value in endoleak embolization, as it allows for controlled hardening under the appropriate conditions (e.g., alginate compositions remaining a liquid until it is in the presence of a divalent cation such as calcium or barium). Liquid embolic agents, e.g., alginate embolic agents such as EmboGel, can be used to quickly and safely embolize endoleaks. Specifically, such compositions can be used for the treatment of Type II endoleaks in patients with Abdominal Aortic Aneurism (AAA).

In aspects, the invention also provides catheters, compositions, and methods for reversible blockage of non-vascular conditions such as treatment of nasal passages in case of epistaxis, treatment in the fallopian tubes as a reversible contraceptive or potentially useful for in vitro fertilization, or other nonvascular conduit in body, for example the bronchi.

Bone Related Conditions

Also treated by the methods of the invention are bone related diseases or disorders. For example, in embodiments, the biocompatible compositions (e.g., hydrogels) can be impregnated with osteogenic factors or cells into vertebrae for the treatment of osteoporosis. Osteoporosis is a disease that makes bones weak and more likely to break. Although osteoporosis is commonly associated with older women, anyone can develop osteoporosis. As many as half of women and a quarter of men older than 50 will break a bone due to osteoporosis. Risk factors include, but are not limited to, old age, low body weight or body mass index, family history of osteoporosis, low bone mass, and certain medications.

In related embodiments, the biocompatible composition (e.g., hydrogel) is first seeded with a patient's own mesenchymal stem cells (MSCs). A small sample of marrow can then be harvested from a patient and the MSC population is then selected, expanded, and then differentiated into osteoblasts. The differentiation of the MSCs into the osteogenic lineage is achieved by incubating cells with factors such as dexamethasone, ascorbic acid and beta-glycerophosphate.

Neoplastic Conditions

The present invention relates to treating a subject suffering from a neoplastic growth. In embodiments, the method involves the step of administering to the subject a composition comprising a biomaterial as described herein (e.g., alginate polymer). In related embodiments, the method involves the step of administering a compound that reverses biomaterial polymerization (e.g., an alginate lyase, optionally a divalent metal chelator, and the like). In embodiments, the subject has previously received treatment with a biocompatible composition (e.g., alginate hydrogel).

In exemplary embodiments of the methods, the biomaterial composition comprises one or more anti-cancer agents. The anti-cancer agent can be any therapeutic known in the art. Non-limiting examples of anti-cancer agents include any of the following: abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

A neoplastic growth can be any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In embodiments, the invention provides catheters, compositions, and methods for embolizing tumors. For pulmonary tumors, the embolization of tumors is often limited by pulmonary shunts. With dissolvable/reversible biomaterial compositions (e.g., alginate polymer in use with alginate lyase), embolization is possible in patients with previously unacceptable shunts.

Thermal Ablation Therapy

In embodiments, the invention can be used for thermal ablation of tumors. Targeted intratumoral delivery of biomaterial compositions (e.g., EmboGel) containing iron oxides in conjunction with an apparatus for creating an alternating magnetic field can be used for thermal ablation. Thermal ablation (or radiofrequency thermal ablation) relates to heating tumors so hot that the tumor cells die. In the procedure, the tumors are located with ultrasound, computed tomography (CT), magnetic resonance (MR) imaging devices, and the like. Then, the patient is essentially turned into an electrical circuit by placing grounding pads on the thighs. A small needle-electrode with an insulated shaft and an uninsulated distal tip is inserted through the skin and directly into the tumor. Ionic vibration at the needle tip leads to frictional heat. After 10 to 30 minutes of contact with the tumor, the radiofrequency energy kills a sphere of cancer cells, often approximately 2.5- to 5-cm in size. The dead cells are not removed, but become scar tissue and eventually shrink. RFA continues to play a time-tested, major role in the treatment of patients with painful osteoid osteomas in the bone and heart arrhythmias. In addition, RFA has been used to treat painful trigeminal neuralgia for 25 years. Today, the mainstream applications of RFA are increasing. In particular, this minimally invasive, percutaneous technique is showing promise as a treatment option for patients with primary or metastatic liver cancer. More information of thermal ablation is readily available to the public on the World Wide Web, for example at clinicalcenter.nih.gov/drd/tumortherapy.htmL.

Thermochemical ablation and thermal ablation alone can be employed as a treatment for an endless number of well circumscribed malignancies as described above, and at a variety of locations including, but not limited to, brain, liver, breast, ovaries, prostate, stomach, colon, pancreas, cervix, uterus, lungs, bladder, and skin. In addition, thermochemical ablation or thermal ablation may be employed to selectively kill non-malignant tissue as in the case of cardiac ablation.

In embodiments, cardiac thermochemical ablation is performed by administering to a subject biocompatible compositions (e.g., EmboGel or EmboCaps) containing cardiotoxic compounds either directly in the polymer (e.g., alginate) layer or incorporated in liposomes. Cardiotoxic compounds include but are not limited to mitomycin A, mitomycin C, doxorubicin, and anthracyclines.

In related embodiments, to treat Atrial fibrillation and atrial flutter, AV Nodal reentry tachycardia (AVNRT), Accessory Pathways, Ventricular Tachycardia treatment would involve the process of first delivering the biocompatible material (e.g., EmboGel or EmboCaps) through targeted delivery using a catheter as described herein to the appropriate cardiac location. Once the biocompatible material (e.g., EmboGel or EmboCaps) is in place, an AMF generator is applied in the case of iron oxide containing biocompatible material (e.g., EmboGel or EmboCaps) to cause locoregional heating. In the case of gold containing biocompatible material (e.g., EmboGel or EmboCaps), high field focused ultrasound or laser excitement can be employed after delivery of the biocompatible material (e.g., EmboGel or EmboCaps) to the targeted location to cause particle heating.

In addition to cancer ablation, and cardiac ablation, such techniques may be employed to deliver local heating or local heating/drug release in any malignant or non-malignant tissue in the body.

In addition to providing MR detectability, iron oxides can be employed for thermal ablation therapy. Specifically, when exposed to an alternating magnetic field (AMF), iron oxides in chemospheres heat. Non-drug loaded biocompatible material (e.g., EmboGel or EmboCaps) can be utilized for thermal ablation after particle delivery. Drug-loaded EmboGel/EmboCaps can be utilized to simultaneously release drug while heating nearby cells. This thermalchemical ablation strategy may enable greater tumor kill than a purely chemical or thermal approach alone.

The potential of hyperthermia and thermal ablation in cancer therapy has been well noted. Temperatures between 42° C. and 46° C. lead to inactivation of normal cellular processes, whereas above 46° C., extensive necrosis occurs. However, the inability to deposit effective doses of heat in tumor without applying similar heat to nearby normal tissue has prevented widespread clinical use. Difficulties in predicting thermal dose, or obtaining accurate in situ measurements, have been additional problems. New technology is needed to deliver heat selectively to tumor cells and provide predictive dosimetry. Iron oxide loaded chemospheres are useful in such applications.

Particle heat output, or specific absorbtion rate (SAR), is a function of AMF field amplitude. In accordance with previous reports the lowest AMF amplitude (Oe) and highest duty (“on” time) combination—that is, 700 Oe (56 kA/m) and 90% duty—that was tested delivered safely the highest calculated total heat delivered (THD) and was associated with the greatest therapeutic effect on the tumors. However, high amplitudes at this frequency also deposit more nonspecific heat to normal tissues from increased eddy current production. To prevent overheating in normal tissues, the duty must be reduced at these higher amplitudes, providing greater “off” time between pulses for heat to dissipate. By contrast, lower-amplitude AMF can be sustained with little “off” time without compromising safety as the nonspecific heat that is generated in normal tissue does not challenge normal mechanisms that dissipate heat. Consequently, the THD to the tumor can be safety enhanced because the particles generate heat for a greater percentage of the total treatment time despite the decreased SAR. The result is a greater net heat deposited to the tumor and less heat deposited to surrounding tissues.

Therefore, in aspects, the invention provides catheters, compositions, and methods for the selective dissolution of an occlusion in a subject. In embodiments, the methods involve administering to the subject a biocompatible composition (e.g., alginate and optionally a divalent metal chelator) to the first targeted area, thereby providing selective dissolution of an occlusion in the subject. In embodiments, the subject has previously received treatment with an biocompatible composition (e.g., alginate based biomaterial).

In aspects, the methods of the invention are useful for selective dissolution of an occlusion that occurs in a vessel not targeted for treatment. For instance, in some cases, alginate biomaterial has been found to protrude out of the neck of the aneurysm and migrate into the parent artery during injection, a situation that carries a high risk of major complication, such as vessel occlusion and stroke. Similar complications may result from the use of alginate in other therapeutic indications, such as in inadvertent obliteration of a normal cerebral artery during the embolization of a vascular malformation. Administration of alginate lyase and the divalent metal chelator according to the methods of the invention are useful in eliminating alginate biomaterial in unwanted locations. In exemplary embodiments, administration of the alginate lyase and the divalent metal chelator occurs after occlusion, for example immediately after the unwanted occlusion, 5 minutes, 10 minutes, 30 minutes, 1 hour, 2 hours, 5 hours, 10 hours, 15 hours, 24 hours, 48 hours, or more. Additional biocompatible systems capable of reversal/dissolution are known in the art. The ordinary artisan would readily understand how to utilize these alternative polymer systems in the methods described herein.

Bulking or Remodeling

In aspects, the invention provides methods for selective control of bulking or remodeling in a subject. In embodiments, the methods involve administering to the subject a biocompatible material (e.g., an alginate based biomaterial) to a targeted area. In embodiments, a composition that dissolves the biomaterial (e.g., alginate lyase) is further administered to the subject at the targeted area, thereby permitting selective control of bulking or remodeling in the subject. In related embodiments, such compositions further comprise a divalent metal chelator.

For certain applications, self-polymerizing alginate is used. For example, a self-polymerizing agent can be used in for cosmetic bulking procedures. A self-gelling alginate is described in US publication 2006/0159823, which is hereby incorporated by reference in its entirety herein.

In embodiments, a self-gelling alginate can be modified to contain an optical agent to assess localization. For example, the FDA approved optical agent inodcyanine green has used at a concentration of 0.005% to assess localization after injection with infrared. Gels can be made by mixing a solution of sodium alginate (Protanal SF 120) and a calcium alginate dispersion (Protaweld TX 120). The amount of calcium alginate can be 1.5%, and the amount of sodium alginate can be 1%. The solution and dispersion are mixed and 5 mL of the gel injected into a 50 mL conical tube. The sample was left to gel for 1 hour. After complete hardening, 0.5 mL of alginate lyase, as described herein, was added to the sample causing complete dissolution.

One of ordinary skill in the art will readily understand how to use EmboClear in the various embodiments described herein. See, e.g., US patent 2006/0159823.

In addition to indocyanine green, Feridex, Gold dextran50 (Nanocs), Barium sulfate solution, PFOB micelles, PFCE micelles, and the like can be added (e.g., up to a concentration of 20% vol/vol) to the pregelled alginate and gellation will still occur. Further the contrast containing gels could be dissolved with EmboClear.

In embodiments, the methods are used to treat urinary incontinence.

In embodiments, methods are in a subject that is undergoing plastic or reconstructive procedures. Biocompatible materials (e.g., alginate based biomaterials) can be used as a bulking agent for plastic and reconstructive procedures. Compositions to dissolve the biocompatible material (e.g., alginate lyase compositions) can further be used for secondary remodeling and consistency adjustment.

For example, a nonporous sac can first be implanted and then filled with a biocompatible material (e.g., EmboGel). Unlike current surgical procedures, such a procedure can be completed percutaneously as the sac can be placed collapsed percuatenously and then filled percutaneously post implantation with the biocompatible material (e.g., EmboGel). Such a design is particularly attractive for breast and cheek augmentation. In the case of a microporous mesh sac, the biocompatible material (e.g., EmboGel) can also be filled with therapeutic factors and act as a large depot for locoregional drug delivery.

Controlled Release Delivery, Nanoparticles, and Liposomes

The invention also provides catheters, compositions and methods for controlled release of an agent in a subject. In embodiments, the methods involve administering to the subject a biocompatible material (e.g., an alginate based biomaterial) to a targeted area. In embodiments, a composition that dissolves the biomaterial (e.g., alginate lyase) is further administered to the subject at the targeted area, thereby resulting in controlled release of the agent from the biocompatible material. In related embodiments, such compositions further comprise a divalent metal chelator.

As described above, in embodiments, the methods are used to treating a subject suffering from a vascular or non-vascular condition.

Any agent is suitable for use in this method. Exemplary agents include, but are not limited to, chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins, antibiotics, vaccines, photodynamic agents, and analgesics.

In aspects, the agent (e.g., therapeutic agent) is provided as a nanomaterial. In embodiments, the agent is contained within the nanomaterial. In embodiments, the therapeutic agent is bound to the nanomaterial.

A nanomaterial can be, but is not limited to, nanocontainers, biological nanomotors, peptide-based self-assembling materials, nanorobots, smart nanodevices as anticancer therapeutics, nanocomposite devices, nanoparticles comprised of carbohydrates, virus particles, lipids, DNA, dendrimers, microchips, drug-loaded microchips, micropumps, hyperbranched polymers, polymer brushes, nanofibers, polymeric nanotubes, nanocapsules, Biosensors, nanotubes, nanowires, chemical sensors, nanohorns, nanorods, MEMS Micro-Electro-Mechanical systems, fluorescent nanoparticles, magnetic nanoparticles, colloidal gold nanoparticles, colloidal gold biofunctionalized nanomodules, magnetic nanoparticles for magnetic guided ‘tag and drag delivery’, nanoparticles conjugated with biological ligands, metal nanoclusters, dendrimer nanocomposites, DNA-linked nanoparticles, nanocolloids (organosols and hydrosols), metal nanopowders (Ag, Au, Pt, Pd), metal nanoparticles and magnetic fluids, palladium nanoparticles, nanomaterials comprised of silicon, aluminum nitride, zinc oxide, platinum, titanium dioxide, silicon dioxide, silicon carbide, cobalt, carbon (graphite), aluminum oxide, cerium oxide, aluminum, gold, silver, copper, nickel. Nano-glasses, nano-ceramics, Cu alloys, Ni alloys, Zn alloys, Co alloys, Zr alloys, nobel metals, light metals, Ti, Ti—Al, Ti transition metals alloy (Fe or Ni or Cu), Mg—Ni, Fe—Cu—Nb—Si—B alloy, Fe-transition metal alloy (Co, Ni, Cr, Cu, Zr), Al-transition metal alloy (Fe, Ni, Ti, Zr), Mg, Al—Mg alloy.

In embodiments, the nanomaterial is selected from, but is not limited to, microboxes, microchips, microfluidic pumps, magnetic resonance microcoil, quantum dots, antibody targeted nanomaterials, nanocontainers, and nanoboxes.

Nanomaterials can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl₄.

In embodiments, the nanomaterials are gold nanoparticles. Gold nanoparticles not only impart radiopacity to the biocompatible material (e.g., EmboCaps and EmboGel) but also enable visualization. Further, by use of high field focused ultrasound or laser excitement in laser photothermal therapy, the particles and surrounding hydrogel will heat. In cases in which the biocompatible material (e.g., EmboCaps and EmboGel) contains heat sensitive liposomes, this will cause a burst release effect of drug from the biomaterial. Further when a therapeutic factor is directly incorporated into the biomaterial (e.g., alginate component of EmboCaps or EmboGel), heat will increase the porosity of the hydrogel thereby increasing the rate of release.

Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl₄, are well known in the art. For example, the methods described herein as well as those described elsewhere (e.g., US 2001/005581; 2003/0118657; and 2003/0053983, each of which is hereby incorporated by reference) can be used to make nanoparticles.

A nanoparticle can have at least one agent linked to its surface. Any of the agents described herein can be linked covalently, non-covalently, or coordinated to the surface of the nanoparticle. For example, all the bonds from an agent to a nanoparticle can be covalent bonds to the surface of the nanoparticle. In some cases, some of the bonds are covalent to the surface of the nanoparticle, and some are noncovalent to the surface of the nanoparticle. In some cases, some of the bonds are covalent to the surface of the nanoparticle, and some are coordinate to the surface of the nanoparticle. In some cases, all of the bonds are noncovalent to the surface of the nanoparticle.

In embodiments, a nanoparticle can have two, three, four, five, six, or more agents linked to its surface. In related embodiments, many molecules of an agent are linked to the surface of the nanoparticle at many locations. Accordingly, in embodiments, when a nanoparticle is described as having, for example, two agents linked to it, the nanoparticle has two distinct agents, each having its own unique molecular structure, linked to its surface. In some cases, one molecule of an agent can be linked to the nanoparticle via a single attachment site or via multiple attachment sites.

An agent can be linked directly or indirectly to a nanoparticle surface. For example, an agent can be linked directly to the surface of a nanoparticle or indirectly through an intervening linker. Any type of molecule can be used as a linker For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In cases where the nanoparticle includes gold, a linker can be any thiol-containing molecule. Reaction of a thiol group with the gold results in a covalent sulfide (—S—) bond. Linker design and synthesis are well known in the art. Any type of agent can be linked to a nanoparticle. For example, an agent can be a therapeutic agent that has a therapeutic effect in the body. Examples of therapeutic agents include, without limitation, anti-angiogenic agents, chemotherapeutic agents, anti-inflammatory agents, anti-bacterial agents, anti-fungal agents, growth factors, immunostimulatory agents, anti-cholinergic agents, insulin, and insulin analogs.

A therapeutic agent can be in any physical or chemical form, including an antibody, an antibody fragment, a receptor, a receptor fragment, a small-molecule, a peptide, a nucleic acid, a peptide-nucleic acid, and the like. A therapeutic agent can function as a targeting agent in addition to functioning as a therapeutic agent. A targeting functionality can allow nanoparticles to accumulate at the target at higher concentrations than in other tissues. In embodiments, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function.

In embodiments, the nanoparticle has a diagnostic agent linked thereto. In embodiments, a diagnostic agent and a therapeutic agent can both be linked to a nanoparticle. A diagnostic agent can allow the imaging of a nanoparticle in vivo. For example, a patient administered a nanoparticle having a diagnostic agent and a therapeutic agent linked thereto can be imaged once, e.g., to locate and/or stage a tumor, or at multiple time points, e.g., to monitor the efficacy of the therapeutic agent.

Any type of diagnostic agent can be linked to a nanoparticle, including, for example, an MR imaging agent, a radio-imaging agent, an X-ray imaging agent, a near-IR imaging agent, and the like. Two or more diagnostic agents can also be linked to a nanoparticle, such as an MR imaging agent and an X-ray imaging agent, or a near-IR imaging agent and an MR imaging agent. An MR imaging agent can be a metal chelate, e.g., can include a chelating ligand and a paramagnetic metal ion coordinated thereto. Any type of chelating ligand can be used, including cyclic and acyclic chelating ligands such as DTPA, DOTA, DOTMA, DTPA-BMA, DOTAGA, and HP-DO3A. Examples of paramagnetic metal ions include, without limitation, Gd(III), Fe(III), Mn(II), Cr(III), Cu(II), Dy(III), Ho(III), Er(III), Eu(III), Tb(II), Tb(III), and Tb(IV).

In aspects of the invention, the agent is incorporated into a liposome.

Liposomes are formed when phospholipids and their derivatives are dispersed in water. Upon dispersion in water the phospholipids form closed vesicles called “liposomes”, which are characterized by lipid bilayers encapsulating an aqueous core. Various liposomes have been used as carriers for entrapped therapeutic agents, such as drugs, enzymes and genetic sequences for use in medical science, in pharmaceutical science and in biochemistry.

Examples of liposome compositions include U.S. Pat. No. 4,983,397; 6,476,068; 5,834,012; 5,756,069; 6,387,397; 5,534,241; 4,789,633; 4,925,661; 6,153,596; 6,057,299; 5,648,478; 6,723,338; 6,627218; U.S. Pat. App. Publication Nos: 2003/0224037; 2004/0022842; 2001/0033860; 2003/0072794; 2003/0082228; 2003/0212031; 2003/0203865; 2004/0142025; 2004/0071768; International Patent Application Nos. WO 00/74646; WO 96/13250; WO 98/33481; Papahadjopolulos et al., Proc Natl Acad Sci U.S.A. 88: 11460-11464 (1991); Allen et al., Semin Oncol 31: 5-15 (2004); and Weissig et al., Pharm. Res. 15: 1552-1556 (1998), each of which is hereby incorporated by reference in its entirety.

Examples of lipid compositions containing targeting factors include U.S. Pat. Nos. 5,049,390; 5,780,052; 5,786,214; 6,316,024; 6,056,973; 6,245,427; 6,524,613; 6,749,863; 6,177,059; 6,530,944; U.S. Pat. App. Publication. Nos. 2004/0022842; 2003/0224037; 2003/143742; 2003/0228285; 2002/0198164; 2003/0220284; 2003/0165934; 2003/0027779; International Patent Application Nos. WO 95/33841; WO 95/19434; WO 2001037807; WO 96/33698; WO 2001/49266; WO 9940789; WO 9925320; WO 9104014; WO 92/07959; EP 1369132; JP 2001002592; Iinuma et al., Int J Cancer 99:130-137 (2002); Ishida et al., Pharmaceutical Research 18:1042-1048 (2001); Holmberg et al., Biochem. Biophys. Res. Comm. 165:1272-1278 (1989); Nam et al., J. Biochem. Mol. Biol. 31:95-100 (1998); and Nag et al., J. Drug Target 6:427-438 (1999), each of which is hereby incorporated by reference.

A variety of agents may be included in the lipid-containing compositions of the present invention, for example, any agent described herein, including genetic material. In embodiments, the agent can be a therapeutic such as an anticancer agent, for example, an anticancer agent suitable for encapsulation in a liposome. The amount of agent to be included in the lipid-containing compositions, and formulations thereof, as described herein can be readily determined by the skilled artisan in view of the teaching herein provided and taking into account factors specific to the intended use, the specific agent, and the individual to be treated, as described further herein. In certain embodiments, the agent may be a nucleic acid, for example, but not limited to, antisense oligonucleotides, ribozymes, and the like.

The lipid-containing compositions described herein can be modified with targeting factors and directed to a particular target cell. The term “targeting factor” refers to a moiety that can bind to a receptor or a surface antigen present on the surface of a target cell. In embodiments, the targeting factors are directed to cell surface receptors on a particular target cell. In embodiments, the targeting factor is a protein or a peptide that can be attached to a lipid component of the lipid-containing composition. In related embodiments, targeting factors are selected such that the targeted receptor or antigen is present only on cells that are targeted for the delivery of the agent or labeled compound (e.g., pathogenic cells) and not present on healthy cells. In related embodiments, a greater number of receptors or antigens are expressed on the target cells (e.g., pathogenic or diseased cells) compared to non-targeted (e.g., healthy) cells.

In embodiments, the receptor or antigen that binds the targeting factor is either not present or present in low numbers on healthy cells such that binding with the targeting factor does not occur with frequency. In other words, targeting factors need to selectively deliver the liposomes as described herein (including encapsulated drug) to the targeted cells (e.g., pathogenic, unhealthy, etc.). Selective delivery of the encapsulated drug to the targeted cells thus reduces the occurrence of adverse effects due to the effect of encapsulated agent or labeled compound on non-targeted (e.g., healthy) cells, thereby also reducing the adverse effects experienced by the individual to whom the composition, or formulation thereof, is administered.

Exemplary targeting factors include, but are not limited to, transferrin, folic acid, folate, hyaluronic acid, sugar chains (e.g., galactose, mannose, and the like), fragments of monoclonal antibodies, asialoglycoprotein, and the like, as well as other targeting factors known to the skilled artisan. In embodiments, the targeting factor is a protein or peptide directed to a cell surface receptor (e.g., transferrin, folate, folic acid, asialoglycoprotein, and the like). In embodiments, the targeting factor is directed to an antigen (e.g., fragments of monoclonal antibodies, including, but not limited to Fab, Fab′, F(ab′)₂, Fc, and the like). It is not intended that targeting factors include intact or whole monoclonal antibodies. The term “whole antibody” or “intact antibody,” and cognates thereof, as used herein generally refer to antibody IgG of immune globulin. A fragment of a monoclonal antibody generally refers to a decomposition product of the monoclonal antibody, for example, a fragment obtained by using protease digestion, such as pepsin, and the like. In embodiments, the targeting factor is not directed to an antigen (e.g., is not a fragment of a monoclonal antibody, e.g., Fab, Fab′, F (ab′)₂, Fc, etc).

In embodiments, the therapeutic liposomes are coated with protein. The protein can be, but is not limited to, antibodies, receptors, and cell surface markers.

In embodiments, the agent is a cell secreting a therapeutic factor. The cell can be any of the following: autogenic or allogenic fibroblasts, endothelial cells, transgenic cells, mesenchymal stem cells, embryonic stem cells, extraembryonic stem cells, embryonic germ cells, cardiac stem cells, umbilical stem cells, cardiac stem cells, all pluripotent and multipotent stem cell sources, pancreatic islet cells, hepatocytes, skin cells, intestinal stem cells, myoblasts, endothelial cells, cardiac myoblasts, dendritic cell, autologous tumor cells (method of sensitization and potential vaccine delivery), Monocyte derived activated killers, Natural Killer T Cells, patients own cancer cells with liposomal I1-2, cultured chondrocytes, hematopoietic stem cells, sertoli cells, xenogenic cell sources of all listed above, skin cells, adipocytes, skin-derived stem cells, neural stem cells, glial progenitor cells, oligodendrocyte and oligo precursors, fat stem cells, other stem cells sources such as from amniotic fluid, baby teeth, bone marrow cells, cord and placental blood, fat tissue, fetal cells, unfertilized ova, pancreas, breast, and the like.

Autogenic or allogenic fibroblasts, endothelial cells or transgenic cells secreting therapeutic factors may be added to the biocompatible material (e.g., alginate) prior to delivery in order to create a bioactive tissue scaffold that may provide tissue regrowth from the inside out. Thus, in embodiments, the biomaterial is linked to an agent such as a tissue scaffold, microcapsules, wound dressings, and the like.

In embodiments, in the case of cardiac thermochemical ablation, biocompatible material (e.g., EmboCaps and EmboGel) can contain cardiotoxic compounds either directly in the polymer layer (e.g., alginate) or be incorporated in liposomes. Suitable cardiotoxic compounds include, but are not limited to, mitomycin A, mitomycin C, doxorubicin, anthracyclines.

Labels and Fiducial Markers

In aspects, the invention provides biocompatible materials containing an agent that is a label. In embodiments, invention provides biocompatible materials containing fiducial markers. The label can be any suitable molecule known in the art, including, but not limited to, contrast agents, quantum dots, antibodies, liposomes, nanoboxes, and the like.

In related aspects, the invention provides methods for the controlled release of a label in a subject. In embodiments, the method involves administering to the subject a biocompatible material (e.g., an alginate based biomaterial) comprising a label. In embodiments, the method involves further administering to the subject a composition that dissolves the biocompatible material (e.g., alginate lyase). In related embodiments, administration of this composition results in controlled release of the label. In certain embodiments when the biomaterial is an alginate based material, the composition that dissolves the biomaterial may further comprise a divalent metal chelator.

In embodiments, the methods are employed to create fiducial markers visible in magnetic resonance imaging, x-ray, ultrasound modalities, and the like.

In embodiments, the methods are employed for diagnostic purposes. For example, the method is suitable for use for selected angiography of a labeled vessel.

The label used in the method of the invention can be any label that is suitable for incorporation into a biomaterial (e.g., an alginate based biomaterial), and for use in, for example, diagnostic purposes. The label can be selected from the group that consists of, but is not limited to, radiolabel, fluorescent label, tissue dye. The label can be contained within a micelle. The radiolabel can be, but is not limited to any one of carbon 14, carbon 14 intermediates, tritium-labeled, iodine 125, and antibody targeted radioisotopes. The fluorescent label can be, but is not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be, but is not limited to, methylene blue.

In embodiments, the label is contained within a liposome. A variety of labeled compounds are suitable for use in the lipid-containing compositions of the present invention. The labeled compound may be an agent useful in carrying out in vivo diagnostic procedures. As with the incorporation of agents as described herein, the amount of labeled compound to be included in the lipid-containing compositions, and formulations thereof, as described herein can be readily determined by the skilled artisan in view of the teaching herein provided and taking into account factors such as the labeled compound selected, the use intended for the composition or formulation, and the individual to be diagnosed, as described further herein. Exemplary labeled compounds include, for example, materials comprising radioisotopes (e.g., ³H, ⁴C, ⁶⁷Ga, ¹¹¹In, ¹²⁵I, ¹²⁵I,), material comprising fluorescent moieties (e.g., fluorescein, fluorescein isothiocyanate, etc.), material comprising enzyme (e.g., peroxidase, alkaline phosphatase, etc.), as well as additional labeled compounds known to those of skill in the art. As will be appreciated by the skilled artisan, the selection of the labeled compound and methods used in diagnosis will depend upon the organ (e.g., liver, pancreas, prostate, etc.), tissue (e.g., malignant or non-malignant or tissue type (e.g., brain, cardiovascular, etc.) to be investigated.

In embodiments, the invention provides methods for the controlled release of a label to mark lesions for radiosurgery, the method involves administering to the subject a biomaterial (e.g., an alginate based biomaterial) linked to a label. In embodiments, the methods further involve administering to the subject a composition that dissolves the biomaterial (e.g., alginate lyase). In related embodiments, administration of the composition that dissolves the biomaterial results in controlled release of the label and marking of the lesion for radiosurgery. In certain embodiments, such a composition may further comprise a divalent metal chelator.

The label can be selected from the group that consists of, but is not limited to, radiolabel, fluorescent label, tissue dye. The label can be contained within a micelle. The radiolabel can be, but is not limited to any one of carbon 14, carbon 14 intermediates, tritium-labeled, iodine 125, and antibody targeted radioisotopes. The fluorescent label can be, but is not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be, but is not limited to, methylene blue. In exemplary embodiments, the label is contained within a liposome.

In related aspects, the invention provides methods for the controlled release of a contrast agent in a subject. In embodiments, the method involves administering to the subject a biocompatible material (e.g., an alginate based biomaterial) comprising a contrast agent. In embodiments, the method involves further administering to the subject a composition that dissolves the biocompatible material (e.g., alginate lyase). In related embodiments, administration of this composition results in controlled release of the contrast agent. In certain embodiments when the biomaterial is an alginate based material, the composition that dissolves the biomaterial may further comprise a divalent metal chelator.

In embodiments, the contrast agent can be, but is not limited to, any magnetic resonance contrast agents, radiopaque contrast agents, ultrasound contrast agents, nuclear medicine imaging contrast agents, and the like that are well known in the art.

Contrast agents can be, but are not limited to, optical agents, PET probe, ultrasound contrast agent, Radioisotopes, magnetic resonance image contrast agent, radiopaque contrast agent for visualization on X-ray modalities, for example DSA, Fluoroscopy, CT, X-Ray, and the like.

As described in detail above, in aspects of the invention, the biocompatible material is delivered at the end of a diagnostic bronchoscopy. Thus, in embodiments, the biocompatible material comprising the agent (e.g., marker) is delivered using a delivery system as described herein, at the end of a diagnostic bronchoscopy.

Thus far the role for intrathoracic fiducial markers is in thoracic malignancies. The ready availability of spiral CT scanners and increased number of scans of the chest are detecting many more lung nodules, including clinically T1 N0 lung cancers. Many patients with these mostly incidentally asymptomatic small cancers, however, have cardiopulmonary comorbid conditions such that they are not surgical candidates for standard lobectomies. Alternatives that have developed include radiofrequency ablation, conformal intensity modulated radiation, gamma-knife beam, proton-beam radiation, and surgically minimally invasive VATS for sublobar resections. With the exception of radiofrequency ablation that is a CT-guided transthoracic procedure, fiducial markers placed adjacent to the often fluoroscopically invisible target lesion are needed or desirable to guide various treatments. Markers such as contrast agents or wires may be delivered transcutaneously but require a separate CT-guided transthoracic procedure that entails the risk of pneumothorax in a high-risk group.

Therefore, in embodiments, the invention provides methods for delivering agents (e.g., markers) at the end of a diagnostic bronchoscopy. In embodiments, the agent is barium that is catheter delivered via the working channel of bronchoscopes.

In embodiments, the agent is a colorimetric dye visible through the pleura. In related embodiments, the methods are used guide VATS wedge resection.

In embodiments, the methods provide bronchoscopic delivery of radiopaque metallic coils to guide VATS and of the delivery of gold seeds, pellets and coils initial developed for intratumoral deposit in other organ types such as the prostate gland to guide high-dose external beam radiation. This is performed by pushing out of the working channel of the bronchoscopes radiopaque markers of the appropriate caliber.

Embolic Compositions

In aspects, the biomaterials are used as embolic materials. In embodiments, the embolic material is administered to a subject at a target site using a catheter as described herein.

In embodiments, the biomaterials comprise embolic hydrogels that form expanding, swelling stents or space-fillers.

As is readily understood by the ordinary artisan, the embolic compositions described herein are useful in a variety of bioapplications. Nonlimiting examples for such bioapplications include occluding blood vessels, occluding aneurysms, occluding other body lumens such as fallopian tubes, filling aneurysm sacs, as arterial sealants, and as puncture sealants.

In embodiments, the hydrogels are used as treatment for vascular aneurysms in a manner similar to other types of mechanical, embolus generating vasoocclusive devices. In one such procedure, an aneurysm is treated by inserting a stent formed of a hydrogel material into the vessel, and then hydrating and expanding the hydrogel material until the stent occludes the vascular wall, sealing it from the parent vessel. Biodegradable hydrogels have also been used as controlled-release carriers for biologically active materials such as hormones, enzymes, antibiotics, antineoplastic agents, and cell suspensions. U.S. Pat. No. 6,113,629 relates to the use of hydrogels for use in occluding aneurysms, and is incorporated herein by reference in its entirety.

In embodiments, the hydrogels are used as lung reduction therapy.

In embodiments, the hydrogels are used as depot drug release vehicles for treatment of malignant and other pulmonary pathological processes.

Embolization of blood vessels is performed for a number of reasons, e.g. to reduce blood flow to and encourage atrophy of tumors, such as in the liver, to reduce blood flow and induce atrophy of uterine fibroids, for treatment of vascular malformations, such as arteriovenous malformations (AVMs) and arteriovenous fistulas (AVFs), to seal endoleaks into aneurysm sacs, to stop uncontrolled bleeding, or to slow bleeding prior to surgery. An aneurysm (a dilation of a blood vessel) poses a risk to health from the potential for rupture, clotting, or dissecting. Rupture of an aneurysm in the brain causes stroke, and rupture of an aneurysm in the abdomen causes shock.

Gynecologic embolotherapy may be conducted for a variety of purposes including the treatment of uterine fibroids, the treatment of postpartum and post caesarean bleeding, the treatment of post surgical vaginal bleeding, the prevention and/or treatment of hemorrhage from ectopic pregnancy, prophylactically prior to myomectomy and in obstetrical patients at high risk for bleeding, such as those patients with placenta previa, placenta accreta, uterine fibroids, and twin fetal death.

Embolics have also been used to treat cancer. For example, embolics can be used to occlude the vasculature feeding a solid tumor. As an adjunct to embolization, drug-loaded embolics, such as drug-loaded microspheres, have been used for in situ delivery of chemotherapeutic agents and/or therapeutic agents designed to treat inflamed or diseased tissue. In addition, clinicians have administered chemotherapeutic agents in combination with embolic PVA particles. This type of regional therapy may localize treatment at the site of the tumor, and therefore the therapeutic dose may be smaller than the effective systemic dose, reducing potential side effects and damage to healthy tissue.

One of ordinary skill in the art would readily understand how to adjust the formulation, route of delivery, and the like, based on disclosures provided herein and taking into account general information about the particular application (see U.S. Pat. Nos. 7,311,861 and 6,676,971, relating to liquid embolics and polymer-based particles and embolic microspheres, each of which is hereby incorporated by reference).

Tissue Scaffolds and Dressings

In aspects, the invention provides methods for the selective dissolution of a wound dressing in a subject. In embodiments, the method comprises the steps of administering a biomaterial (e.g., alginate) based wound dressing to a wound. In embodiments, the method comprises waiting a period of time, for example, waiting a time of 1 day, 2 days, 3 days, 4 days, 5 days, 6 days, 7 days, 10 days, 12 days, 14 days, 21 days, 15 days, 28 days, 30 days, 35 days, 40 days, or more days and then administering a composition that dissolves the biomaterial (e.g., alginate lyase, and optionally a divalent metal chelator) to the wound. In related embodiments, administration of the that dissolves the biomaterial provides selective dissolution of the wound dressing in the subject. In embodiments, the process of administering the biomaterial, followed by a waiting period, and then followed by administration of the composition that dissolves the biomaterial is repeated until the wound is healed.

In embodiments, the compositions of the invention could effectively dissolve the hydrogel component of the commercially available alginate dressings, for example, but not limited to ALGISITE M, SEASORB, CONTREET-H, INVACARE Calcium Alginate Wound Dressing, NUDERM Alginate Wound Dressing, CURASORB Calcium Alginate Dressing, TEGAGEN HG Alginate Dressing, KALTOSTAT Alginate Dressing, ALGISITE M Calcium Alginate Dressing, RESTORE Calcium Alginate Dressing, and RESTORE Silver Calcium Alginate.

Calcium alginates have long been known for their ability to form fibers or nonwoven materials. These have been used primarily as swabs or dressings for medical, surgical or other purposes, such as described in European Patent Specification, EP 0721355 B1, entitled “Alginate Wound Dressings, which is incorporated herein by reference in its entirety. Supplied in the form of nonwoven wound dressings for the treatment of exudating wounds, the calcium alginate dressing is said to encourage the formation of controlled ion-active gel over the wound site, which reacts with the sodium ions in the exudate. Examples of exudative wounds include pressure ulcers, venous stasis ulcers, diabetic ulcers, arterial ulcers, second degree burns and skin graft donor sites. The alginate based wound dressing can be a solid dressing, more specifically a solid wound dressing comprised of an alginate based biomaterial, capable of delivering an effective wound-healing agent. U.S. Pat. No. 7,112,320 discloses solid wound dressings, including solid wound dressings based on calcium alginate, capable of delivering an effective wound healing amount of fibronectin to a wound site, and is incorporated herein by reference.

In embodiments, the alginate based wound dressing further comprises one or more therapeutic agents. The therapeutic agent can be any agent known to be useful in treating wounds. Such agents include, but are not limited to, an antibiotic, such as cephalosporins, macrolides, penicillins, quinolones, sulfonamides, tetracycline, aminoglycosides, lincomycin, chloramphenicol, glycopeptides, monobactams, carbapenems, carbacephems, metronidazole, antitubercular, antileprotics, oxazolidinones, ketolides, an analgesic, an antifungal, an antiviral, enzymes, vaccines, gene delivery vectors, such as liposomes, cationic lipids, lentiviral vectors, antibodies, hormones, and recombinant glycoproteins.

In embodiments, the methods are used to burn victims, where it is desirable to have a wound dressing that does not have to be removed, but rather dissolves away. The method of the selective dissolution of a wound dressing mitigates the pain and skin damage that occurs with bandage removal.

In embodiments, selectively dissolvable alginate dressings can be dissolved with EmboClear.

An alginate dressing, termed Smart Skin, is a dressing for split-thickness skin graft. Similar to ALLEVYN (Smith & Nephew), Smart Skin has a hydrophilic inner layer consisting of a collagen and calcium alginate mixture. Applied to the hydrophilic inner layer is an outer polyurethane waterproof film layer that prevents bacterial contamination and maintains a moist wound environment. Smart Skin provides a unique advantage over Allevyn as the inner hydrogel layer can be selectively dissolved with EmboClear. This overcomes the major drawback of Allevyn, namely its propensity to strongly adhere to the wound bed causing mechanical trauma to the newly formed delicate epithelium when the dressing is changed.

Smart Skin can be impregnated with nanocrystalline silver particles (10 nm from Nanocs) by directly dissolving the alginate at a concentration of 2% w/v in a 0.01% Ag aqueous solution prior to polymerization. In embodiments, larger silver nanoparticles are preferable (20-50 nm Nanocs). Additionally, collagen, hyaluronic acid or an alternate biodegradable biomaterial may be added to the silver alginate solution prior to polymerization with calcium or an alternate divalent cation. In embodiments, in addition to directly incorporating silver nanoparticles in the inner alginate layer, an alternate formulation is provided on the outer layer, consisting of a silver-coated high-density polyethelene mesh similar to Acticoat (Smith and Nephew).

In embodiments, alternate compounds are incorporated into the alginate matrix of Smart Skin to promote keratinocyte growth, including, but not limited to, M4 agonists, M3 antagonists, basic fibroblast growth factor (bFGF), keratinocyte growth factor (KGF), WNTs, and Keratinocyte growth factor-2 (KGF-2). These agents may be directly incorporated into the alginate layer prior to polymerization or may first be entrapped in liposomes that are then added to the liquid alginate layer prior to polymerization. This unique combination of liposome impregnated hydrogel scaffold ensures a slow release of hydrophilic compounds as demonstrated by the release of doxorubicin from liposomes in EmboCaps previously described in detail in WO/2008/127290.

In embodiments, alginate can act as a component of a full-thickness skin scaffold. In embodiments, alginate is combined with other biomaterials such as collagen, hyaluronic acid or PEGDA. In related embodiments, EmboClear can be added to selectively dissolve the alginate component of the scaffold. This will ease removal of an infected tissue scaffold or alternatively would give the clinician selective control over the porosity of the scaffold thereby facilitating tissue ingrowth.

In embodiments, the skin scaffold is seeded with a number of cell sources.

In embodiments, the dressing or scaffold comprises an agent that is a cell secreting a therapeutic factor. The cell secreting a therapeutic factor can be any suitable cell, including, but not limited to, autogenic or allogenic fibroblasts, endothelial cells, transgenic cells, mesenchymal stem cells, embryonic stem cells, extraembryonic stem cells, embryonic germ cells, umbilical stem cells, pluripotent and multipotent stem cells, endothelial cells, dendritic cell, hematopoietic stem cells, sertoli cells, xenogenic cell sources of all listed above, skin cells, adipocytes, skin-derived stem cells, neural stem cells, glial progenitor cells, oligodendrocyte precursors, oligo precursors, fat stem cells, other stem cells sources such as from amniotic fluid, baby teeth, bone marrow cells, cord blood, placental blood, fat tissue, fetal cells and breast.

### Diagnostics

The biomaterials (e.g., alginate based biomaterials) can be combined with magnetic resonance imaging and/or ultrasound contrast agents, in order to provide visibility during procedures performed with these imaging modalities. The visibility of the biomaterials can be set to persist on a long-term basis, or to decrease after administration at a pace that can be controlled.

In embodiments, the biomaterial composition is formulated to provide transient radio-opacity and long-term magnetic resonance (MR) signal. The embolic material would thus be optimally radio-opaque for safe delivery at the time of the therapeutic procedure, have its radio-opacity decrease shortly after injection in order to avoid beam-hardening artifacts on follow-up CT studies, while retaining MR signal for long-term non invasive follow up imaging studies.

MR contrast agents such as the iron-based agents Feridex and Endorem, the gadolinium-based agents such as Omniscan and Magnevist, and the fluorinated magnetic resonance (MR) contrast agents such as perfluorocarbon and perfluoropolyether, can all be used in conjunction with the biomaterials described herein.

In embodiments, the biomaterial comprises bromofluorocarbons, which provide Hotspot imaging on 19F MR imaging, and have sufficient radio-opacity to be conspicuous on CT.

In embodiments, the biomaterial comprises barium or bismuth sulfate, which are suitable for long term labeling and imaging with standard clinical fluoroscopic equipment. This type of labeling is useful as radio-opaque markers for subsequent radiotherapy.

In embodiments, the biomaterials described herein are used in diagnostic applications for selected angiography of a particular vessel.

### Pharmaceutical Compositions

In aspects, the biomaterials are provided as a pharmaceutical composition. In embodiments, the pharmaceutical compositions comprise an active ingredients. In embodiments, these pharmaceutical compositions contain suitable pharmaceutically acceptable carrier, diluent, or excipient. In embodiments, the pharmaceutical compositions are placed in an appropriate container and labeled for treatment of an indicated condition with information including amount, frequency and method of administration.

In embodiments, the compositions of the invention include a biomaterial (e.g., an alginate based biomaterial). In related embodiments, also provided is a composition with a clearing agent (e.g., alginate lyase, and optionally, divalent metal chelators) that can selectively dissolve the biomaterial. Together, the compositions have potential use for a variety of clinical and experimental applications.

The compositions of the biomaterial and clearing agent can be used generally to treat a variety of diseases or conditions, can be used as a standalone embolic or bulking material, or it can be combined with various bioactive agents (such as chemotherapeutic agents, radio-isotopes, genes, and the like), or it can be built into a delivery agent with a controllable release (e.g., liposomes).

In embodiments, the compositions are alginate based biomaterial in combination with an alginate lyase clearing agent. Alginate biomaterials can be selectively dissolved after application, using the alginate lyase or any alginate clearing solution. The final product of the dissolution comprises a biocompatible molecule. Thus, the compositions can be used for selective release of bioactive agents, in remote locations and at a controlled pace, such as chemotherapeutic agents, radioisotopes, genes, and the like.

In embodiments, the compositions are used as a bulking agent for plastic and reconstructive procedures. The combination with a clearing agent (e.g., alginate lyase) composition allows for secondary remodeling and consistency adjustment.

The compositions are also suitable for use in any of the methods described herein.

In embodiments, the compositions comprise bioactive agents, such as chemotherapeutic, anti-inflammatory, or antimicrobial drugs, hormonal therapy agents, plasmid or adenovirus for gene therapy applications, stem cells delivery, and the like. All these agents may be combined with any procedure described herein. The compositions can also be used for the delivery of radiolabeled particles for loco-regional radiotherapy.

Cleavable components of alginate can be incorporated into an endless number compounds, for example propylene glycol alginate, allowing for selective degradation. Further, to achieve proper viscosity, elasticity and porosity designer alginates can be used.

In embodiments, the invention provides for compositions comprising an alginate lyase and a divalent metal chelator. According to the invention, the divalent metal chelator is a proteinaceous or a non-proteinaceous metal chelator. In embodiments of the method, the divalent metal chelator is a calcium chelator. In embodiments, the divalent metal chelator can also be, but is not limited to, EDTA, DTPA, DMSA, citrate, tartrate, dimercaptol, penicillamine, deferoxamine, dithizone, cisplatin, and chlorophyll. In certain, the composition comprises very low levels of EDTA, or no EDTA, to maximize cytoxicity.

In embodiments, the compositions of the invention comprise alginate lyase. In related embodiments, the alginate lyase is a bacterial alginate lyase. Bacterial alginate lyases are described by Wong T Y et al., Annual Review of Microbiol 54:289-340 (2000), incorporated herein by reference in its entirety. In embodiments, the bacterial alginate lyase is selected from the group consisting of: Flavobacterium, Flavobacterium, Burkholderia, Corynebacterium, Klebsiella, Photobacterium, Pseudoalteromonas, Pseudomonas, Rhodopirellula, Saccharophagus, Sphingomonas, Streptomyces, Vibrio, and Aspergillus. In embodiments, the composition comprises a Flavobacterium bacterial alginate lyase.

In embodiments, the alginate lyase is a transgenic alginate lyase.

In embodiments, the alginate lyase, or biologically active fragment thereof, comprises the amino acid sequence of SEQ ID NO: 1, or a fragment thereof.

In embodiments, the compositions comprise a contrast agent. Accordingly, the contrast agent can be selected from, but not limited to, magnetic resonance contrast agents, radiopaque contrast agents, ultrasound contrast agents, and nuclear medicine imaging contrast agents. In embodiments, the magnetic resonance contrast agent is selected from, but not limited to, any of: Manganese Oxide, perfluorocarbons, Feridex, Gadolinium, Combidex, Bang Magnetic Particles, Gd-DTPA, Gadolinium And Manganese Derivatives, Superparamagnetic Iron Oxide Particles, gadopentetate dimeglumine, Gd-DOTA, Gd-DTPA-BMA, Gd-HP-DO3A, Gd-DTPA-BMEA, Gd-DO3A-butrol, Gd-BOPTA, Mn-DPDP, Gd-EOB-DTPA, Gd-BOPTA, AMI-25, SH U 555A, gadoflourine-M, AMI-227, EP-2104R, P947, Gd-DTPA mesophorphryn, SH U 555 C, NC-100150, MS-325, gadoflourine-M, gadomelitolm manganese chloride, ferric ammonium citrate, and barium sulfate suspensions.

In embodiments, the compositions comprise more than one biocompatible materials. In embodiments, a portion of the biocompatible material does not dissolve when treated with a clearing agent (e.g., alginate lyase). Such biocompatible materials can be, but are not limited to, polyvinyl alcohol, sodium polyacrylate, acrylate polymers, hyaluronase polymers, collagen membrane, Porous HA/TCP ceramic composite, hydroxyapatite bone cement, PVP/PMMA, tricalcium phosphate, hydroxyapatite coated collagen fibers, calcium sulphate, hydroxyapatite (HAp), phosphorylcholine (PC), silicone, ultrahigh molecular weight polyethylene, polyethylene, acrylic, nylon, polyurethane, polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly(lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids), poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, methylacrylate, para-xylylene, biodegradable copolymers, copolymer surface coatings, starch polymers, polylactic acid, cellophane, tyrosine polycarbonates lactide and glycolide polymers, collagen, PTFE, silicone, keratin-based materials, fibrous composites—carbon fiber and particles, polymer composites, artificial/natural material composites, glass-ceramic/metal composites, glass-ceramic/nonmetal composites, dental composites, ormocer, hydrogels, timed-release foams, and polymeric carriers.

In embodiments, the compositions of the invention encompass an alginate based wound dressing. The alginate based wound dressing can comprise one or more therapeutic agents. The therapeutic agent can be, but is not limited to, an antibiotic, an analgesic, an antifungal, and an antiviral.

The compositions of the invention can contain an alginate biomaterial. The alginate biomaterial can comprise D-mannuronic acid and D-guluronic acid. The alginate biomaterial can comprise an alginic acid. In exemplary embodiments, the alginate biomaterial is alginate.

Alginate for use in the compositions on the invention can be obtained from, but not limited to, any of the following: Macrocystis, Laminaria, Ascophyllum, Chlorophyceae, Phaeophyceae, Rhodophyceae, and Cyanophyceae. In embodiments, the alginate is obtained from Aminaria hyperborean. In embodiments, the alginate is obtained from Laminara digita. In embodiments, the alginate is obtained from Ascophyllum nodosum. In embodiments, the alginate is a bacterial alginate. In embodiments, is obtained from a heterotrophic bacteria. In related embodiments, the heterotrophic bacteria are selected from the group consisting of: Pseudomonadaceae and Azotobacteriaceae.

In embodiments, the compositions of the invention comprise divalent cations. The divalent cation is selected from, but not limited to, Ca²⁺, Mg²⁺, Ba²⁺ and Sr²⁺. In embodiments, the cation is Ca²⁺. The divalent cation can be, in other embodiments, a synthetic compound with divalent orientation. In embodiments, the divalent cations are administered in liposomes or microbubbles. Liposomes can be, but are not limited to heat sensitive liposomes, ultraviolet sensitive liposomes and ph sensitive liposomes. The divalent cation can be administered simultaneously with the biomaterial, or after administration of the biomaterial.

In embodiments, the composition comprises one or more anti-cancer agents. Anti-cancer agents can include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, vinflunine, and the like. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

It will be appreciated by those of skill in the art that the clearing agent (e.g., alginate lyase compositions) can be used for dissolving the biomaterial (e.g., alginate) in vivo in a number of applications already introduced or currently reaching a clinical phase. The ability provided by the clearing agent to selectively dissolve biomaterial in a controlled manner offers ways to transport diagnostic and therapeutic agents in remote locations using biomaterial-based vectors and release them where and when needed. In addition, the clearing agent compositions can potentially be implemented in various biomaterial applications currently explored or in clinical use. Such applications include for example (i) nerve regeneration scaffold (peripheral and spinal cord), (ii) soft tissue augmentation, for instance in use as space filler for plastic surgery and to treat stress urinary incontinence, (iii) chondrocyte scaffold, (iv) encapsulation of cellular therapeutics, (v) drug delivery capsules, (vi) embolic agents as microspheres, (vii) wound dressing for split-thickness burns, and the like.

### Dosage and Mode of Administration

The invention provides catheters and compositions for carrying out the methods described herein. In embodiments, the biomaterial compositions (including clearing agent compositions) described herein are delivered to a target site in a subject using a catheter as described herein. In embodiments, the duel lumen catheter is positioned in the lumen of an endoscope or is incorporated into the design of the endoscope. The distal end of the duel lumen catheter is positioned so as to deliver the biocompatible material to the targeted treatment area in a minimally invasive manner.

By way of example, a patient suffering from or susceptible to various vascular and non-vascular lesions as described herein can be treated as follows. EmboGel, EmboClear or a EmboGel/EmboClear therapeutic combination can be administered to the patient, preferably in a biologically compatible solution or a pharmaceutically acceptable delivery vehicle, by injection with a catheter as described herein.

In embodiments, the biomaterials are polymerized outside of the body and implanted after they have been crosslinked (e.g., alginate with a divalent cation). In embodiments in which the biomaterial only partially contains alginate, rigidity may not result from gelation in divalent cation. For these reasons, alginate containing biomaterials may have the consistency appropriate for injection/delivery in the absence of administering a metal cation, such as a divalent cation. The dosages administered will vary from patient to patient.

In any of the treatments described, a therapeutically effective dosage regimen should be used. By “therapeutically effective”, one refers to a treatment regimen sufficient to restore the subject to the basal state, as defined herein, at the cellular or tissue site of manifestation or to prevent brain edema in an individual at risk thereof or restore the subject's brain to the basal state. Alternatively, a “therapeutically effective regimen” may be sufficient to arrest or otherwise ameliorate symptoms of brain edema. In embodiments, an effective dosage regimen requires providing the medication over a period of time to achieve noticeable therapeutic effects.

A therapeutic composition of use in the invention can be given in a single- or multiple doses.

In embodiments, a therapeutic composition of the invention will be administered in a single dose. Biomaterial compositions (e.g., alginate) may be provided in the range of 1 nanoliter per kg body weight to 50 mL per kg body weight. The clearing agent (e.g., alginate lyase) may be provided in the range of 1 nanoliter per kg body weight to 50 mL per kg body weight. In embodiments, The alginate lyase and divalent metal chelator are administered at a ratio of between 99:1-1:99, for example a ratio of 99:1, 98:2, 97:3, 96:4, 95:5, 90:10, 85:15, 80:20, 75:25, 70:30, 60:40, 50:50, 40:60, 30:70, 25:75, 20:80, 15:85, 10:90, 5:95, 4:96, 3:97, 2:98, 1:99.

The dosages of alginate composition and alginate lyase composition will be administered at different time points, depending on the method of treatment, as considered appropriate by the treating physician.

In embodiments, the therapeutic composition is administered in multiple doses. A multiple dose schedule is one in which a primary course of administration can include 1-10 separate doses, followed by other doses given at subsequent time intervals required to maintain and or reinforce the level of the therapeutic agent. Such intervals are dependent on the continued need of the recipient for the therapeutic agent, and/or the half-life of a therapeutic agent. The efficacy of administration may be assayed by monitoring the reduction in the levels of a symptom indicative or associated with brain edema which it is designed to inhibit. The assays can be performed according to methods known to one skilled in the art.

A therapeutically effective regimen may be sufficient to arrest or otherwise ameliorate symptoms of a disease. An effective dosage regimen requires providing the regulatory drug over a period of time to achieve noticeable therapeutic effects wherein symptoms are reduced to a clinically acceptable standard or ameliorated. The symptoms are specific for the therapeutic use.

The biomaterials can be delivered in a number of ways according to the instant methods. Alginate can be injected concurrently with a solution of calcium, or any divalent cation, and the hardening (gelation) of the alginate based biomaterial can be achieved through the addition of calcium-containing liposomes. Such liposomes can be forced to release their calcium-containing contents through heat or light activation. Using such technique would result in the polymerization of alginate biomaterial through heat-sensitive or photosensitive liposomes, respectively. According to the invention, any other natural or synthetically derived compounds with two adjacent positive charges may be used in place of calcium for gelation of the alginate based biomaterial.

In particular embodiments of the invention, a single syringe can be used to deliver the alginate based biomaterial and divalent cation compositions. A single syringe with two compartments of varying size attached to the same plunger handle is used to deliver an exact ratio of alginate and divalent cations. This method of delivery enables consistent delivery of the proper ratio of the alginate and calcium chloride compound. U.S. Patent Application No. 20050133046 describes delivery of alginate based biomaterials, and is incorporated by reference herein in its entirety.

In another exemplary method of delivery, any of the compositions of the invention described herein can be administered with the catheters described herein in combination with an endoscope.

In a further exemplary method of delivery, any of the compositions of the invention described herein can be delivered using a bronchoscope. Use of a bronchoscope according to the methods of the invention is suited, in exemplary embodiments, for use in pulmonary applications. A bronchoscope can be used to deliver any of the compositions of the invention inside the bronchial tree and airway. Further, the delivery mechanism has use in the method of tumoral marking for subsequent radiotherapy, using, for example, any of the methods of the invention described herein. In particular, delivery of the compositions of the invention with a bronchoscope has use in the methods of controlled release of a label in a subject, or controlled release of an agent in a subject. Further, the method of delivery has application in lung volume reduction procedures using any of the compositions of the invention as described herein.

In embodiments, the composition comprising alginate lyase and the divalent metal chelator are co-administered from the same catheter. The device for administration can be a syringe. Alternately, a microcatheter is suitable for administration.

Localized Administration

Localized administration of a therapeutic composition according to the invention is preferably by injection directly in to blood vessels or by means of a microcatheter, drip device, drug pump or drug-saturated solid matrix from which the composition can diffuse implanted at the target site.

In embodiments, the therapeutic composition according to the invention may be used to deliver radiolabeled particles. Such use is particularly suited for the delivery of radiolabeled particles for locoregional radiotherapy.

Systemic Administration

Systemic administration of a therapeutic composition according to the invention may be performed by methods of whole-body drug delivery are well known in the art. These include, but are not limited to, intravenous drip or injection, subcutaneous, intramuscular, intraperitoneal, intracranial and spinal injection, or by the use of an implantable, time-release drug delivery device.

Systemic administration is advantageous when a pharmaceutical composition must be delivered to a target tissue that is widely-dispersed, inaccessible to direct contact or, while accessible to topical or other localized application, is resident in an environment (such as the digestive tract) wherein the native activity of the nucleic acid or other agent might be compromised, e.g. by digestive enzymes or extremes of pH.

### Kits or Pharmaceutical Systems

The present compositions may be assembled into kits or pharmaceutical systems for use in dissolving biomaterial (e.g., an alginate based biomaterial). The kits can comprise a clearing agent (e.g., an alginate lyase, and optionally a divalent metal chelator). The kit can further comprise instructions for use.

Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for dissolving the biomaterial with the clearing agent.

In embodiments, the present compositions may be assembled into kits or pharmaceutical systems for use in treating a subject that has received treatment with an alginate based biomaterial. The kits can comprise an alginate lyase, a divalent metal chelator, and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for treating a subject that has received treatment with an alginate based biomaterial.

In embodiments, the present compositions may be assembled into kits or pharmaceutical systems for use in treating a subject suffering from a vascular or non-vascular condition, a vascular or non-vascular occlusion, a vascular or non-vascular hemorrhage, or a neoplastic growth, optionally wherein the subject has previously received treatment with a biomaterial (e.g., an alginate based biomaterial). The kits can comprise a clearing agent (e.g., an alginate lyase and optionally a divalent metal chelator) and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for treating a subject suffering from a vascular or non-vascular condition, a vascular or non-vascular occlusion, a vascular or non-vascular hemorrhage, or a neoplastic growth, optionally wherein the subject has previously received treatment with an alginate based biomaterial.

In embodiments, the present compositions may be assembled into kits or pharmaceutical systems for selective dissolution of an occlusion in a subject. The kits can comprise a biomaterial and clearing agent (e.g., an alginate based biomaterial, alginate lyase, a divalent metal chelator), and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for selective dissolution of an occlusion in a subject.

In embodiments, the present compositions may be assembled into kits or pharmaceutical systems for selective delivery of a therapeutic agent to a targeted non-occluded vessel in a subject, or selective control of bulking or remodeling, or the controlled release of a label in a subject, or the controlled release of a label to mark lesions for radiosurgery in a subject, or the controlled release of a contrast agent in a subject. The kits can comprise a biomaterial and clearing agent (e.g., an alginate based biomaterial, alginate lyase, a divalent metal chelator), and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for selective delivery of a therapeutic agent to a targeted non-occluded vessel in a subject, or selective control of bulking or remodeling, or the controlled release of a label in a subject, or the controlled release of a label to mark lesions for radiosurgery in a subject, or the controlled release of a contrast agent in a subject.

In embodiments, the present compositions may be assembled into kits or pharmaceutical systems for the selective dissolution of a biocompatible material in a subject. The kits can comprise a biomaterial and clearing agent (e.g., an alginate based biomaterial, alginate lyase, a divalent metal chelator), and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for selective dissolution of a biocompatible material in a subject.

In embodiments, the present compositions may be assembled into kits or pharmaceutical systems for selective dissolution of a wound dressing in a subject. The kits can comprise a biomaterial and clearing agent (e.g., an alginate based biomaterial, alginate lyase, a divalent metal chelator), and instructions for use. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention for selective dissolution of a wound dressing in a subject.

In any of the above embodiments, the kits can further comprise a catheter as described herein. In related embodiments, such kits can further comprise instructions for using the catheter to administer the pharmaceutical compositions to a targeted site in the subject.

Having now generally described the invention, the same will be more readily understood through reference to the following Examples, which are provided by way of illustration, and are not intended to be limiting of the present invention, unless specified.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Sulfonated Alginate Preparations

As previously described in WO/2008/127290, which is hereby incorporated by reference in its entirety, when a therapeutic agent(s) is incorporated in sodium alginate, it is trapped within the calcium matrix when exposed to the divalent cation calcium. In unmodified alginate, small molecular weight drugs are rapidly eluted from the hydrogel. To improve upon this system, alginate was modified to include sulfonate groups that effectively bind charged drug thereby increasing drug retention and facilitating prolonged release

Alginate can be sulfated to facilitate ionic binding of doxorubicin, irinotecan or other similarly charged drugs. For example, chlorosulfonic acid and formamide were mixed with glucomannan powder at 40° C. After 5 hours of stirring (500 r/min), 95% ethanol was added to precipitate the crude product, which was filtered and washed with water until it became neutral, yielding sulfated glucomannan. Sulfated glucomannan can be added to the below alginate preparation in varying concentrations to facilitate binding of charged therapeutic compound such as doxorubicin.

Methods for preparing alginate based biomaterials, including alginate lyase compositions, are well known in the art. See, e.g., WO/2008/127290. For example, alginate based biomaterial can be prepared as follows. PRONOVA UP MVM alginate from FMC Biopolymers (Haugesund, Norway) was added at a concentration of 2% w/v to a standard nonionic contrast agent (Iohexyl, Omnipaque 300, Amersham Health, Princeton, N.J.), diluted 1:1 in normal saline (0.91% w/v of NaCl) or to a normal saline solution with 0.25 mg/mL tantulum powder (Advanced Materials), and added for contrast. In addition 0.01% w/v methylene blue was added to the alginate formulations to enable colorimetric identification of polymer on histological analysis post biopsy. Polymerization of the various formulations was achieved by co-injection with a 100 mM calcium chloride solution with alginate in a 4 French inner catheter (Infinity™, Cordis Corporation, Miami, Fla.) and calcium delivered through a 5 French outer catheter (Envoy, Cordis Corporation, Miami, Fla.).

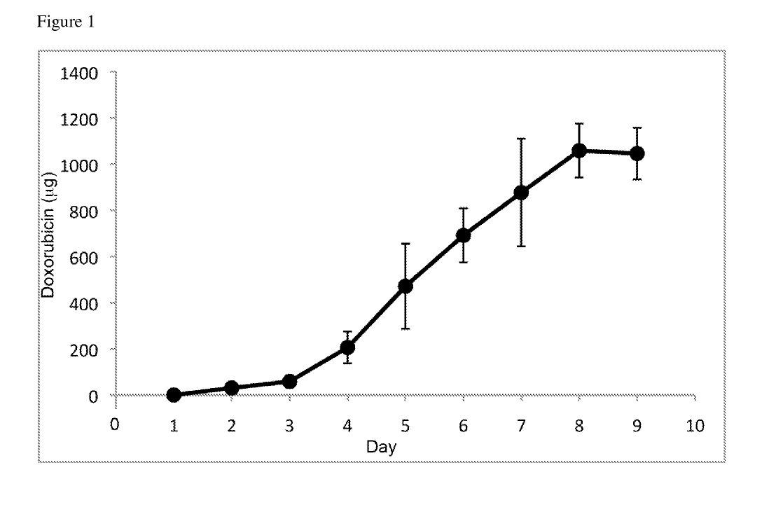

For examination of doxorubicin release from glucomannan alginate blends, sulfated glucomannan as described above was added 1:1 with PRONOVA UP MVM alginate from FMC Biopolymers (Haugesund, Norway), and at a concentration of 2% w/v to a standard nonionic contrast agent (Iohexyl, Omnipaque 300, Amersham Health, Princeton, N.J.). To this 6 mg doxorubicin was added and the mixture was polymerized in 100 mM calcium chloride. Dox loaded hydrogels were then placed into dialysis tubing (molecular weight cut-off of 10,000 DA) sealed at both ends with clips. Dialysis tubing was then placed into a beaker containing 1000-fold excess Hepes Buffered Saline with 30% FBS and incubated at 37° C. and pH 7.4. A doxorubicin standard curve ranging from 0 to 6 mg/mL drug was prepared in saline. From these standard curves the percentage of total drug release was calculated. Doxorubicin release was monitored by measuring the time dependence of the absorbance at 485 nm. Release profile is described in FIG. 1.

### Example 2

### Endobronchial Injection of Alginate

Experiments were performed on healthy swine (40-45 kg). Animals were sedated with 1 mL/50 lbs of telazol/ketamine/xylazine (100/10/100 mg/mL IM) and induced with sodium thiopental (25 mg/mL IV to effect). All CT examinations were performed two hours after alginate injection using a multidetector CT scanner with imaging parameters: 130 kV, 100 mAs, 1.0 s, pitch 2, slice thickness 500 μm, image size 512×512 (Somatom Volume Zoom, Siemens, Erlangen, Germany). In order to evaluate the in vivo detectability of the alginate, the scanning range covered the diaphragm to the pelvic floor.

Diagrams of the catheter are provided in FIGS. 2-4.

XperCT

An angiographic system (Allura Xper FD 20; Philips Medical Systems, Best, the Netherlands) was used. For each CCT acquisition, the area of interest was positioned near the system isocenter and scanned with the “propeller” movement through 240°. The scan time was 20 seconds, depending on the number of acquired images. A series of 620 images were collected at a frame rate of 30 frames per second. Each frame had a matrix size of 1024×1024 with a depth of 14 bits. The system transferred the projection image data to a workstation in parallel to the acquisition for volume data reconstruction using commercially available software (XperCT Release 1; Philips Medical Systems; Best, the Netherlands).

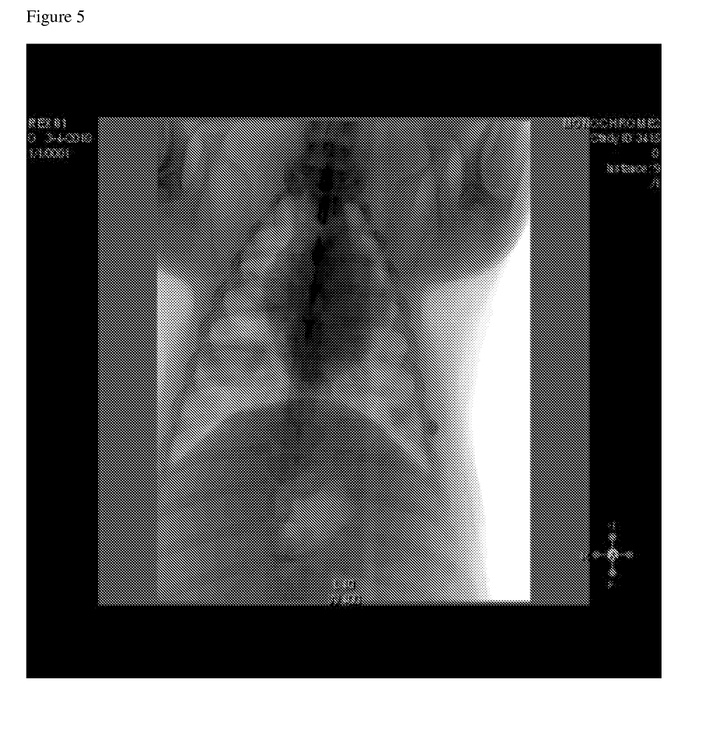

FIG. 5 provides an Xper CT image of 1 mL of barium alginate delivered with a concentric catheter through the working channel of an endoscope. The results showed a well localized radiopaque mass in the lower lung field.

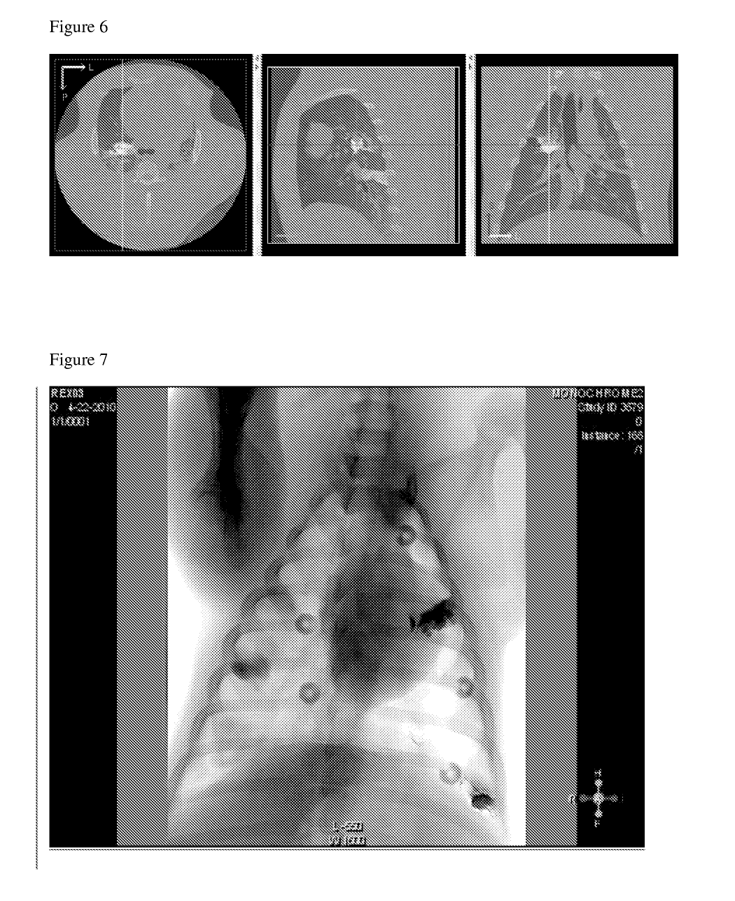

As shown in FIG. 6, biomaterial can also be delivered and visualized in the upper lung field.

As showing in FIG. 7, delivery and imaging can be accomplished simultaneously in both the upper and lower lung fields.

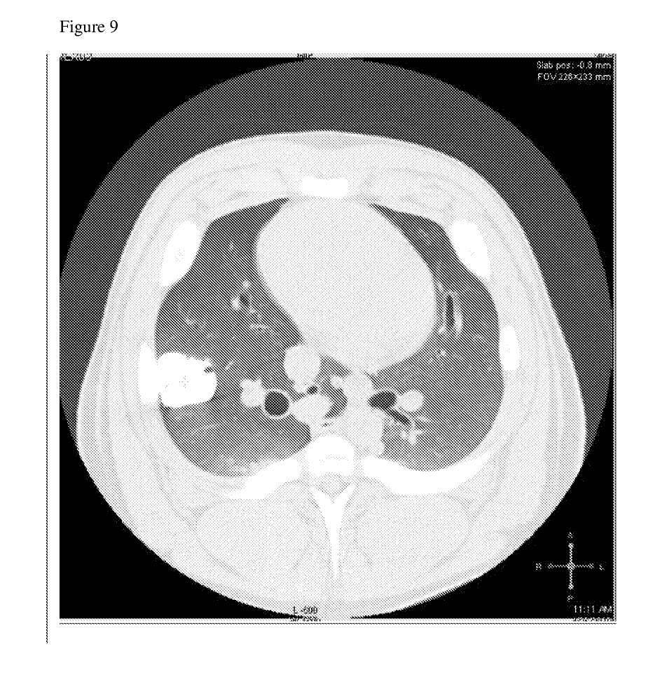

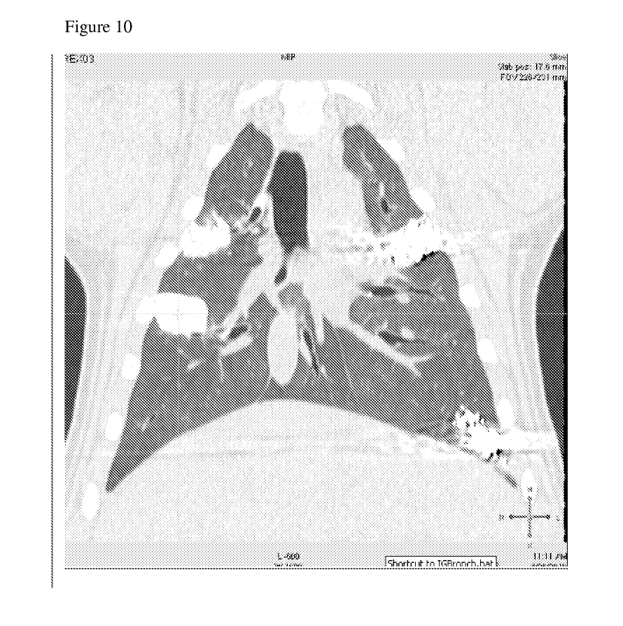

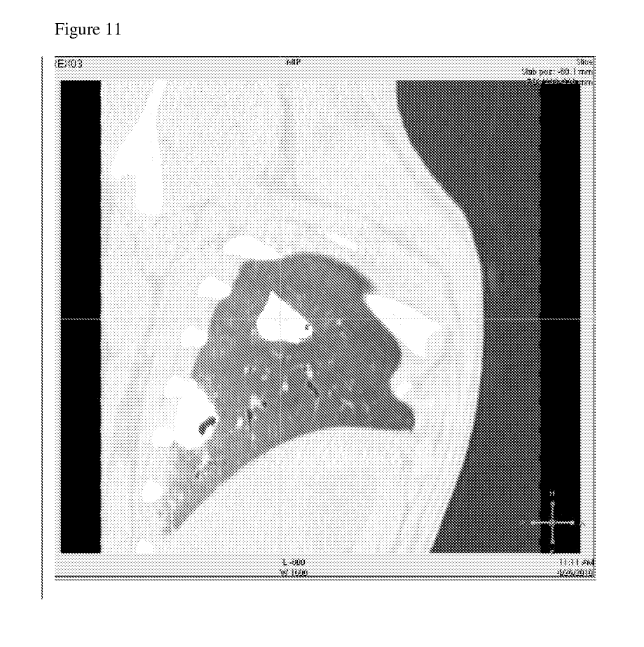

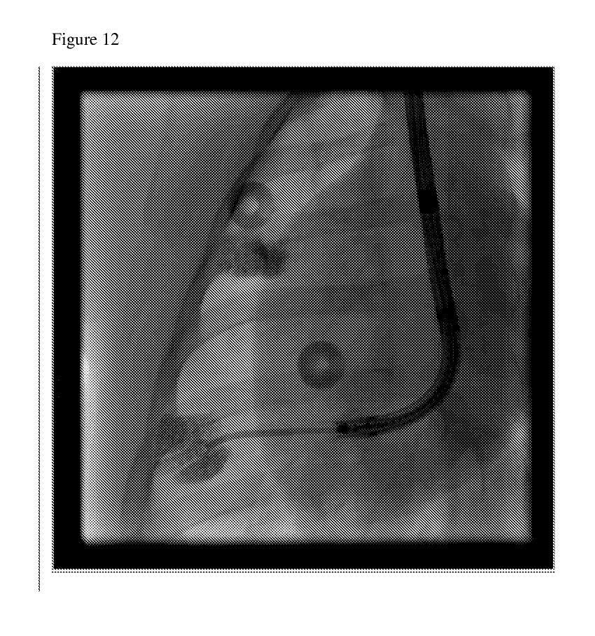

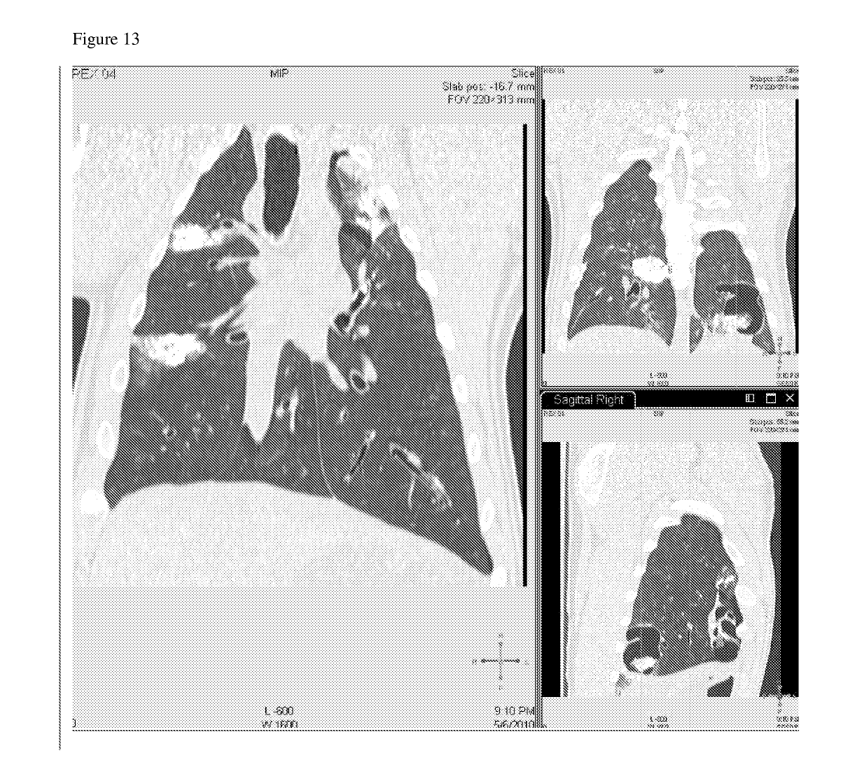

Additional images of treated animals are provided in FIGS. 9-11 and 13. These images demonstrate that the biomaterial compositions of the present invention can successfully be delivered to a target site in a subject using the catheters described herein.

As reported Tsushima et al. Resp Medicine 100:737-745 (2006), the diagnostic yield for standard (non-CT-augmented) X-ray fluoroscopy guided biopsy procedures, in the peripheral lung field, for lesions with diameter<10 mm, 11-15 mm, and 16-20 mm, were 7.7%, 20%, and 64.3%, respectively. On average, for lesions<20 mm, Tsushima et al. had a diagnostic yield of 30%. With the addition of CT-augmentation to the X-ray guided procedure, diagnostic yield of the procedure was significantly improved.

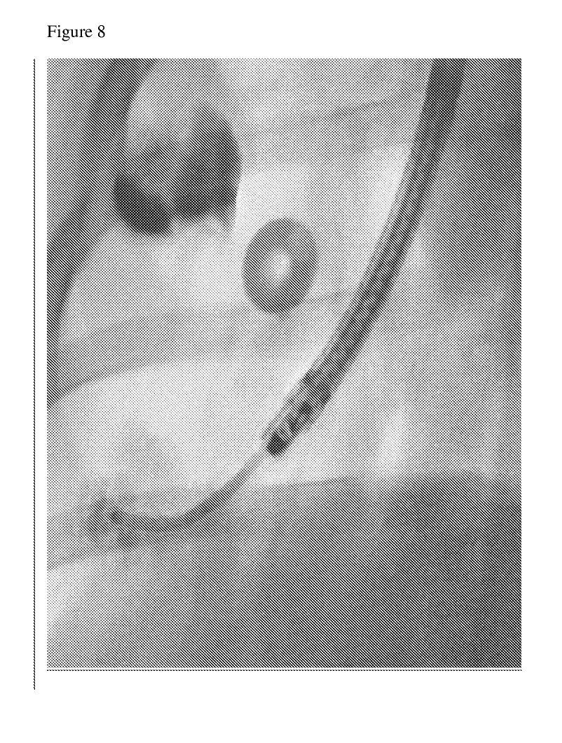

Specifically, under X-ray guidance, radio-opaque targets were inserted into the peripheral lung structure to simulate a peripheral lung cancer nodule (FIGS. 8 and 12). Targets were inserted into both left and right lung. Later, after CT imaging, CT-augmented targeting was attempted to reach these targets with increased accuracy and with better success at biopsy (higher diagnostic yield). A comparison was be made between the ability of the pulmonologist to reach the targets with and without CT-augmentation during bronchoscopy. Hence, two trials were conducted with each animal (left and right lung fields). The distance between various biopsy instruments (needle or aspirator) and the target was measured via additional X-ray imaging. It was found that CT-augmented procedures were closer to the target, increasing the success rate and diagnostic yield of the biopsy procedure by a factor of 2 for lesions<20 mm in diameter.

### Example 3

### Drug Release

Formulations of sodium alginate containing iohexyl and loaded with either doxorubicin or cisplatin were examined for drug elution after crosslinking with calcium chloride. Such formulations have use in the methods described herein, including as a depot formulation for locoregional delivery through endoscopes. Such a formulation enables treatment of nonresectable pulmonary malignancies, or GI malignancies or preoperative reduction in tumor burden.

The mannuronic and gluronic acid moieties of alginate bind cisplatin and doxorubicin, thereby allowing for sustained delivery. Alginate was further modified by sulfonation to facilitate drug binding. Specifically, alginate can be sulfated to facilitate ionic binding of doxorubicin, irinotecan or other similarly charged drugs. Chlorosulfonic acid and formamide were mixed with glucomannan powder at 40° C. After 5 hours of stirring (500 r/min), 95% ethanol was added to precipitate the crude product, which was filtered and washed with water until it became neutral, yielding sulfated glucomannan. Sulfated glucomannan was added to the above-described alginate preparation in varying concentrations to facilitate binding of charged therapeutic compound such as doxorubicin.

For example, 8 mg of cisplatin/1 mL alginate or 8 mg of doxorubicin/1 mL alginate in the base alginate polymer were prepared. After a homogenous suspension is created by serial passage of alginate between syringes connected to a three-way stopcock, the drug loaded alginate material was delivered as previously described for the non-drug loaded gels. Varying the amounts of drug per volume of alginate controlled the dose of total alginate delivered.

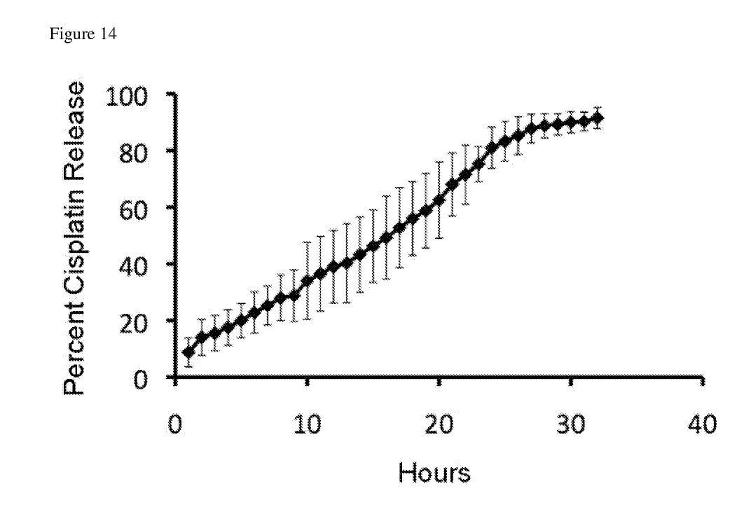

To evaluate release of drug from the biomaterial, 7 mg of cisplatin was added to a solution of sodium alginate, PRONOVA UP MVM alginate from FMC Biopolymers (Haugesund, Norway) 5% w/v, and standard nonionic contrast agent (Iohexyl, Omnipaque 300, Amersham Health, Princeton, N.J.) (1 mL, 5% w/w). The drug loaded solution was then crosslinked with 100 mM calcium chloride solution. The solution was decanted, and the gel was dried by air. The cisplatin loaded gels (7 mg cisplatin/mL) were incubated in phosphate-buffered saline (PBS, 1 mL) in tubes at 37° C. Over a period of 32 hours, 5 microliter samples of the solution were taken. Spectrophotometeric measurements at 230 nm of the sample were measured using a NanoDrop 1000 spectrophotometer (Thermo Scientific). Absorbances were measured against a standard cisplatin curve of absorbances to determine % of drug release. The results are shown in FIG. 14.

The results described herein demonstrate the effectiveness of advances in designs of concentric catheter hubs. In one embodiment of the invention, two separate catheter hubs are incorporated to accept the syringe containing sodium alginate and the syringe containing calcium chloride. Instead of two separate catheter hubs a single concentric catheter hub would be optimal to allow for use of a concentric syringe system. The concentric syringe would be pre-filled with the appropriate volume and concentration of calcium chloride to polymerize the preloaded volume of alginate. Therefore a dual plunger applied to the syringe would co-inject both alginate and calcium chloride. For particular applications a power injection system would be optimal to ensure steady flow of alginate and calcium chloride. The constant force applied by the power injection system would overcome the resistance inherent in such a design.

Additional advances in the design as described herein include a dual lumen catheter with at least one inflatable balloon cuff that isolates the area of embolic material within a lung segment. An addition channel with conduit solely to the balloon alone is included to allow for inflation with a suitable liquid such as saline or alternatively air.

Yet further advances described in detail herein include modifications to the catheter size to create embolic materials of varying shapes and sizes. The size of embolic materials can be adjusted by changing the size of the outer catheter as the size of the precut strand is dependent on the outer diameter of the catheter. The concentric catheter design can be modified so that the internal delivery channel 305 is centrally placed or eccentrically placed within the wall of the outer dual lumen catheter 307.

As also described in detail herein, such catheters can be used in conjunction with any suitable biomaterial, including those described in detail herein. In addition to the use of calcium alginate, any two solutions that rapidly polymerized when mixed could be delivered through a similar catheter design to form embolic particles.

Accordingly, by providing a catheter which allows the components (e.g., the sodium alginate and calcium chloride) from being combined until they reach the distal end of a catheter, the viscosity of the fluids becomes less problematic, thereby lessening the likelihood of flow obstructions in a microcatheter. Furthermore, the amount of time required for the hydrogel to reach the targeted treatment site can be greatly decreased. Finally, by utilizing a catheter treatment sites can be more precisely targeted which in turn allows for more effective treatments.

The results reported herein were obtained using the following methods and materials.

### Animals

Swine (30-50 kg, 12 in total) were premedicated with Ketamine 22 mg/kg, Sedazine 0.9 mg/kg and Telazol 1.1 mg/kg I.M. Since the experiments involved the use of the animal's airway, sedation with isofluorane was not feasible. Instead, the animals were administered continuous intravenous infusions of Propofol. During all procedures, ECG was continuously monitored. An IV catheter was placed in the ear vein for infusion throughout the day of experiments.

### Target Implantation Procedure

In the animal X-ray fluoroscopy suite, the animals were anesthetized as described above and prepped in a standard sterile manner for a bronchoscopic procedure. Under the guidance of a flexible bronchoscope (5 mm Pentax EB-1570K), the bronchoscopist randomly selected paths and reached the smallest possible airway. A mixture of bio-gel with radio contrast (Barium Sulfate Microspheres) and colorimetric marker (Methylene Blue) was loaded onto an implantation catheter using a syringe. The radiopaque mixture was targeted and simulated “lesions”. The loaded catheter was then passed through a 2 mm working channel of the bronchoscope until the tip of the loaded catheter was visible bronchoscopically. Under fluoroscopic guidance, the loaded catheter was advanced beyond the visual range of the bronchoscope until an increase in resistance was felt and the airway was pushed away from the bronchoscope. The loaded catheter was then withdrawn slightly and the syringe depressed, pushing the radio-opaque material out of the catheter's tip and depositing it in a peripheral airway and lung parenchyma. This procedure was repeated in multiple airway segments and in multiple lobes to create multiple “lesions”. All equipment except that for intubation was removed. For validation purposes, fluoroscopic images were recorded immediately after implantation.

### Pre-Operative Imaging Procedure

Once the target materials were in place and the bronchoscope was removed, the animals were transported for CT imaging. Fiducial markers (N≧3) were placed on the chest of the animal after the animal was on the CT table in the supine position. A spiral CT scan (Toshiba, Aquilion One) of the animal was obtained in high-resolution (slice thickness 2 mm, slice increment 1 mm, 120 kilovolt, 325 milliampere). For the purpose of the CT study, the animal was intubated and placed on a ventilator with 100% oxygen. While obtaining the CT scan, ventilation was temporarily stopped at the end of expiration phase for approximately 10-15 seconds. Pre-procedural CT scan images were acquired and saved in DICOM format using a CD. The lesions, airways, and/or other relevant pulmonary structures were segmented and extracted for fusion and visualization.

### Image-Guided Biopsy Procedure

The animals were transported back to the X-ray room for the image-guided biopsy procedure. A Philips Allura Xper FD20/20 fluoroscope system was used for X-ray imaging. The animal was placed on the X-ray table in the supine position. At the beginning of the procedure, two X-ray images of the animal were obtained at different views by changing the primary angle of the C-arm. The X-ray images and the C-arm's angle information were streamed to a workstation via intranet. The workstation was loaded with the preoperative CT image from the CD. The fiducial markers were identified in the X-ray images, allowing the preoperative CT image to be registered to the X-ray system. Intra-operative X-ray images were fused with the pre-operative CT images at the workstation using research software. The projections of airways of interest were overlaid on the intra-operative X-ray images for lesion targeting guidance.

Preparation and anesthesia was performed as described above. The flexible bronchoscope (5 mm Pentax EB-1570K) was advanced towards the ‘target’ using X-ray guidance and possibly the registered CT data. Once reaching the wedge position (where the flexible bronchoscope can no longer be pushed forward), a needle was inserted through the working channel of the bronchoscope and advanced towards the target. Once the needle reached the location closest to the target, X-ray imaging was performed in anterior-posterior and lateral views to confirm that the needle had reached the designated target. The X-ray images were recorded. This procedure was repeated for each target. The radio-opaque material was sampled in the same manner as done during a standard biopsy procedure. The extracted “tissue” was then used for additional confirmation of successful targeting with and without CT augmentation.

The animal will then be euthanized and complete gross examination was performed, with additional microscopic confirmation of lesion location. Heparin was infused to permit better visual, microscopic and photographic confirmation of the simulated lesion targeting procedure. All procedures described were performed within a single day ranging form implantation of targets, to imaging, and back to the image-guided biopsy procedure. The animal was euthanized at the end of the study at the end day.

### Data Analysis

The anterior-posterior and lateral views images were used to confirm needle or forceps position and to measure distance from needle or forceps tip to target. By reading recorded information (primary angle, secondary angle, focal length and patient table translation, etc), the actual 3D distance from needle or forceps tip to target was reconstructed and calculated. The images also aided evaluation for pneumothorax, excessive bending (kinking) of the catheter or any other related complications. Finally, the samples collected from the radio-opaque lesions were examined microscopically to confirm the successful targeting of the lesion. The diagnostic yield was measured for both X-ray guided procedures and CT-augmented X-ray guided procedures as the number of biopsies yielding successful retrieval of radio-opaque material that represents the lesions.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents and publications mentioned in this specification are herein incorporated by reference to the same extent as if each independent patent and publication was specifically and individually indicated to be incorporated by reference.

1. A catheter comprising:
(i) a first delivery channel interdisposed within a second delivery channel, the first delivery channel fluidly communicating a first component of a biocompatible material and the second delivery channel fluidly communicating a second component of the biocompatible material;
(ii) a distal end of the first delivery channel interposed within the second delivery channel, wherein the length of the first delivery channel is shorter than a distal end of the second delivery channel; and
(iii) a mixing tip, wherein the first component and the second component are mixed between the distal end of the first delivery channel and the distal end of the second delivery channel.

2. The catheter of claim 1, wherein the length of the first delivery channel is variable.

3. The catheter of claim 1, wherein the length of the first delivery channel is fixed.

4. The catheter of claim 1, wherein a proximal end of the first delivery channel and the proximal end of the second delivery channel are connected respectively to a syringe via flexible connections.

5. The catheter of claim 4, wherein the syringe comprises
(i) a barrel having a first and second chamber;
(ii) a plunger movably inserted into the first and second chambers of the barrel, wherein the plunger is configured to be fitted into each chamber of the barrel respectively;
(iii) a first connection channel having a proximal end connected to the distal end of the first chamber of the barrel; and
(iv) a second connection channel having a proximal end connected to the distal end of the second chamber of the barrel wherein the first connection channel.

6. The catheter of claim 5, wherein the plunger further comprises two movably independent plungers wherein a first plunger is inserted into a first chamber of the barrel and a second plunger is inserted into a second chamber of the barrel.

7. The catheter of claim 5, wherein the plunger further comprises two sections which are fixed together so as to be movably dependent wherein a first section is inserted into a first chamber of the barrel and a second section is inserted into a second chamber of the barrel.

8. The catheter of claim 1, wherein the catheter is a microcatheter.

9. The catheter of claim 1, wherein at least one chamber contains a first biocompatible material and at least one chamber contains a second biocompatible material and/or a divalent cation to be fluidly communicated to the distal end of the catheter.

10. The catheter of claim 1, wherein the catheter further comprises an endoscope.

11. The catheter of claim 10, wherein the catheter is inserted through a lumen of an endoscope.

12. The catheter of claim 10, wherein the catheter is incorporated into a design of the endoscope.

13. A method for treating a subject having a vascular or non-vascular condition, the method comprising:
a. providing a catheter of claim 1;
b. inserting the catheter into the subject near a targeted area; and
c. administering a biocompatible material to the subject with the catheter, thereby treating the subject.

14. The method of claim 13, wherein the vascular or non-vascular condition is selected from the group consisting of: arteriovenous malformation, endovascular repair failure, osteoporosis, neurovascular lesions, telangiectasias, varicoceles, varicose veins, inflammatory lesions, hemorrhage, occlusion, embolism, neoplastic growth, venous disease, and phlebitis.

15. The method of claim 14, wherein the endovascular repair failure is endoleakage.

16-24. (canceled)

25. A method for treating a subject having a neoplastic growth, the method comprising:
a. providing a catheter of claim 1;
b. inserting the catheter into the subject near a targeted area; and
c. administering a biocompatible material to the subject with the catheter, thereby treating the subject.

26-32. (canceled)

33. A method for treating or preventing osteoporosis in a subject, the method comprising:
a. providing a catheter of claim 1;
b. inserting the catheter into the subject near a targeted area; and
c. administering a biocompatible material to the subject with the catheter, thereby treating the subject.

34-60. (canceled)

61. A method for the selective dissolution of an occlusion in a subject, the method comprising:
a. providing a catheter of claim 1;
b. inserting the catheter into the subject near a targeted area;
c. administering a biocompatible material to the subject with the catheter; and
d. administering to the subject a clearing composition that dissolves the biocompatible material, wherein the clearing composition is administered near the targeted area, thereby providing selective dissolution of the occlusion in the subject.

62-64. (canceled)

65. A method for the selective delivery of a therapeutic agent to a targeted non-occluded vessel, the method comprising:
a. providing a catheter of claim 1;
b. inserting the catheter into the subject to the targeted non-occluded vessel;
c. administering a biocompatible material to the subject with the catheter;
d. administering the therapeutic agent to the subject with the catheter; and
e. administering to the subject a clearing composition that dissolves the biocompatible material, thereby providing selective delivery of the therapeutic agent to the non-occluded vessel.

66-68. (canceled)

69. A method for the selective control of bulking or remodeling in a subject, the method comprising:
a. providing a catheter of claim 1;
b. inserting the catheter into the subject near a targeted area;
c. administering a biocompatible material to the subject with the catheter; and
d. administering to the subject a clearing composition that dissolves the biocompatible material, thereby providing selective control of bulking or remodeling in the subject.

70-71. (canceled)

72. A method for lung volume reduction therapy in a subject, the method comprising the steps of:
a. providing a catheter of claim 1;
b. inserting the catheter into the subject near a targeted area;
c. administering a biocompatible material to the subject with the catheter; and
d. administering to the subject a clearing composition that dissolves the biocompatible material, wherein the clearing composition is administered near the targeted area, thereby providing lung volume reduction therapy in the subject.

73. A method for the controlled release of a therapeutic agent in a subject, the method comprising the steps of:
a. providing a catheter of claim 1;
b. inserting the catheter into the subject to a targeted area;
c. administering a biocompatible material to the subject with the catheter;
d. administering the therapeutic agent to the subject with the catheter; and
e. administering a clearing composition to the subject, thereby providing controlled release of the therapeutic agent.

74-89. (canceled)

90. A method for the controlled release of a label in a subject, the method comprising the steps of:
a. providing a catheter of claim 1;
b. inserting the catheter into the subject to a targeted area;
c. administering a labeled biocompatible material to the subject with the catheter; and
d. administering a clearing composition to the subject, thereby providing controlled release of the label.

91-98. (canceled)

99. A method for the controlled release of a label to mark a lesion for radiosurgery, the method comprising the steps of:
a. providing a catheter of claim 1;
b. inserting the catheter into a subject to a targeted area;
c. administering a labeled biocompatible material to the subject with the catheter; and
d. administering a clearing composition to the subject, thereby providing controlled release of the label in the subject and marking the lesion for radiosurgery.

100-105. (canceled)

106. A method for the controlled release of a contrast agent in a subject, the method comprising:
a. providing a catheter of claim 1;
b. inserting the catheter into the subject to a targeted area;
c. administering a biocompatible material to the subject with the catheter;
d. administering the contrast agent to the subject with the catheter; and
e. administering a clearing composition to the subject, thereby providing controlled release of the contrast agent.

107-110. (canceled)

111. A method for the selective dissolution of a wound dressing in a subject, the method comprising the steps of:
a. providing a catheter of claim 1;
b. inserting the catheter into the subject to a targeted area;
c. administering a wound dressing comprising a biocompatible material to the subject with the catheter;
d. waiting 1 to 14 days; and
e. administering a clearing composition to the subject, thereby providing selective dissolution of the wound dressing in the subject.

112-125. (canceled)

126. The method of any one of claim 61, wherein the clearing composition comprises alginate lyase.

127. The method of claim 126, wherein the method further comprises administering a divalent metal chelator.

128-145. (canceled)

146. A method for delivering a biocompatible material to a subject, the method comprising:
a. providing a catheter of claim 9;
b. inserting the catheter into the subject near a targeted area, wherein the target area is selected from the group consisting of: the gastrointestinal tract, the upper gastrointestinal tract, the lower gastrointestinal tract, the pulmonary tract, the larynx and the upper tracheobronchial tree, the ear, the urinary tract, the female reproductive tract, the abdominal or pelvic cavity, the interior of a joint, and an organ of the chest; and
c. administering the biocompatible material to the subject with the catheter, thereby delivering the biocompatible material to the targeted area.

147. A method for delivering a biocompatible material to a subject during surgery, the method comprising:
a. providing a catheter of claim 9;
b. inserting the catheter into the subject near a targeted area during the surgical procedure; and
c. administering the biocompatible material to the subject with the catheter, thereby delivering the biocompatible material to the targeted area during surgery.

148-169. (canceled)

170. A kit for use in treating a subject having a vascular or non-vascular condition, wherein the kit comprises a catheter of claim 1, and wherein the kit further comprises a biocompatible material.

171. A kit for treating a subject having a vascular or non-vascular hemorrhage, wherein the kit comprises a catheter of claim 1, and wherein the kit further comprises a biocompatible material.

172. A kit for treating a subject having a neoplastic growth, wherein the kit comprises a catheter of claim 1, and wherein the kit further comprises a biocompatible material.

173. (canceled)

174. A kit for the selective dissolution of an occlusion in a subject, wherein the kit comprises a catheter of claim 1, and wherein the kit further comprises a biocompatible material and a clearing composition that is capable of dissolving the biocompatible material.

175. A kit for the selective delivery of a therapeutic agent to a targeted non-occluded vessel, wherein the kit comprises a catheter of claim 1, wherein the kit further comprises a biocompatible material and a clearing composition that is capable of dissolving the biocompatible material, and wherein the kit comprises one or more therapeutic agents.

176. A kit for the selective control of bulking or remodeling in a subject, wherein the kit comprises a catheter of claim 1, and wherein the kit further comprises a biocompatible material and a clearing composition that is capable of dissolving the biocompatible material.

177. A kit for the controlled release of an agent in a subject, wherein the kit comprises a catheter of claim 1, wherein the kit further comprises a biocompatible material and a clearing composition that is capable of dissolving the biocompatible material, and wherein the kit further comprises one or more agents.

178. A kit for the controlled release of a label in a subject, wherein the kit comprises a catheter of claim 1, and wherein the kit further comprises a labeled biocompatible material and a clearing composition that is capable of dissolving the biocompatible material.

179. A kit for the controlled release of a label to mark lesions for radiosurgery in a subject, wherein the kit comprises a catheter of claim 1, and wherein the kit further comprises a labeled biocompatible material and a clearing composition that is capable of dissolving the biocompatible material.

180. A kit for the controlled release of a contrast agent in a subject, wherein the kit comprises a catheter of claim 1, wherein the kit further comprises a biocompatible material and a clearing composition that is capable of dissolving the biocompatible material, and wherein the kit further comprises one or more contrast agents.

181. A kit for the selective dissolution of a biocompatible material in a subject, wherein the kit comprises a catheter of claim 1, and wherein the kit further comprises a biocompatible material and a clearing composition that is capable of dissolving the biocompatible material.

182. A kit for the selective dissolution of a wound dressing in a subject, wherein the kit comprises a catheter of claim 1, wherein the kit further comprises a wound dressing comprising a biocompatible material, and wherein the kit further comprises a clearing composition that is capable of dissolving the biocompatible material.

183. A kit for lung volume reduction therapy in a subject, wherein the kit comprises a catheter of claim 1, and wherein the kit further comprises a biocompatible material and a clearing composition that is capable of dissolving the biocompatible material.

184. A kit for treating or preventing osteoporosis in a subject, wherein the kit comprises a catheter of claim 1, and wherein the kit further comprises a biocompatible material and a clearing composition that is capable of dissolving the biocompatible material.

185-193. (canceled)


SEQUENCE LISTING (3 sequences)

SEQ ID NO: 1 - protein, 368 aa, Pseudomonas aeruginosa
MKTSHLIRIALPGALAAALLASQVSQAADLVPPPGYYAAVGERKGSAGSCPAVPPPYTGSLVFTSKYEGSDSARATLNVKAEKTFRSQIKDITDMERGATKLVTQYMRSGRDGDLACALNWMSAWARAGALQSDDFNHTGKSMRKWALGSLSGAYMRLKFSSSRPLAAHAEQSREIEDWFARLGTQVVRDWSGLPLKKINNHSYWAAWSVMSTAVVTNRRDLFDWAVSEFKVAANQVDEQGFLPNELKRRQRALAYHNYALPPLAMIAPFAQVNGVDLRQENHGALQRLAERVMKGVDDEETFEEKTGEDQDMTDLKVDNKYAWLEPYCALYRCEPNACSRPKKDREPFNSFRLGGEVTRVFSREGGS

SEQ ID NO: 2 - protein, 5 aa, Artificial Sequence
  Description of Artificial Sequence Synthetic peptide
VVVPP

SEQ ID NO: 3 - protein, 5 aa, Artificial Sequence
  Description of Artificial Sequence Synthetic peptide
VPGVG